(19) United States
(12) Patent Application Publication
(10) Pub. No.: US 2026/0061078 A1
(43) Pub. Date: Mar. 5, 2026

(54) Adenoviral-Based In Situ Delivery of Bispecific T Cell Engagers

(71) Applicant: Universität Zürich, Zürich (CH)

(72) Inventors: Jonas KOLIBIUS, Zürich (CH); Patrick Christian FREITAG, Zürich (CH); Andreas PLÜCKTHUN, Zürich (CH); Ronja WIEBOLDT, Basel (CH); Heinz LÄUBLI, Binningen (CH)

(21) Appl. No.: 19/105,122

(22) PCT Filed: Aug. 30, 2023
(86) PCT No.: PCT/EP2023/073807

§ 371 Date: Feb. 20, 2025

(30) Foreign Application Priority Data
Aug. 31, 2022 (EP) 22193151.2

(51) Int. Cl. A61K 48/00; A61K 38/17; A61K 39/00; A61K 39/395; A61P 35/00; C12N 15/86

(52) U.S. Cl. CPC A61K 48/0058; A61K 38/177; A61K 39/39541; A61P 35/00; C12N 15/86; A61K 2039/505; C12N 2710/10043

(57) ABSTRACT

Disclosed herein are recombinant non-oncolytic viruses, for example adenoviruses, that encode bispecific T cell engagers (BiTE's). These BiTE's can be expressed at any desired site of the human body. The non-oncolytic viruses are able to direct expression of the BiTE's in situ, i.e. directly at the site at which the BiTE's exert their action. The non-oncolytic viruses are useful in the treatment of diseases such, as cancer.

### FIELD OF THE INVENTION

Disclosed herein are recombinant non-oncolytic viruses, for example adenoviruses, that encode bispecific T cell engagers (BiTE's). These BiTE's can be expressed at any desired site of the human body. The non-oncolytic viruses are able to direct expression of the BiTE's in situ, i.e. directly at the site at which the BiTE's exert their action. The non-oncolytic viruses are useful in the treatment of diseases such, as cancer.

### BACKGROUND

Even today, about 20 years after they have been generated for the first time, bispecific T cell engagers (BiTE's), receive many attention in the scientific community. By way of their architecture, bispecific T cell engagers form a link between T cells and tumor cells. This causes T cells to exert cytotoxic activity on tumor cells by producing proteins like perforin and granzymes, independently of the presence of MHC I or co-stimulatory molecules. These proteins enter tumor cells and initiate the cell's apoptosis.

Prominent BiTE's include blimatumumab, a CD19/CD3-specific BiTE that is approved for the treatment of certain B cell malignancies, solitomab, a EpCAM/CD3-specific BiTE that is developed for certain types of gynecological cancers, and tebentafusp, a bispecific gp100 peptide-HLA-directed CD3 T cell engager, which is developed for the treatment of certain melanoma.

Despite their clinical success, many unsolved challenges remain. In their typical scFv-scFv format BiTE's are small biomolecules (about 55 kDa in size) and inherently unstable (numerous scFv fragments tend to misbehave if the constant part of the antibody is removed). BiTE's are also filtered out of by the kidney quickly following intravenous injection. Their typical serum half-life is only in the range of a few hours. This leads to challenges to maintain a sufficiently high dose of the drug at the cancerous site.

The present invention provides a solution to these problems by delivering the BiTE's directly to the cancerous site where they can exert their action. This is achieved via the delivery of the BiTE's to the cancerous site via recombinantly engineered adenoviruses.

Various types of adenoviruses are used for therapeutic interventions. Human adenovirus serotype 5 vectors (HAdV-C5) can not only be modified to high-capacity vectors (HC-AdVs) (36 kb packaging size) but are also validated in various clinical trials and animal models (Virus Genes (2017) 53:684-691; J Hematol Oncol (2020) 13:84; Adenoviral Gene Therapy (2002) 7, 46-59). Increasing the safety of human application further, HAdV genomes exist extra-chromosomally, minimizing the risk of unwanted insertional mutation and germline transmission. However, if desired, HC-AdVs have been reported as a single delivery entity combining donor DNA and a Cas9 system, enabling site-specific insertion and deletion (Molecular Therapy: Methods and Clinical Development (2020) 17:441-7), qualifying HAdV-C5 to an ideal and versatile vector.

We hypothesized, that based on our previously described trimeric adapter technology (J Mol Biol (2011) 405:410-26; Proc Natl Acad Sci USA (2013) 110: E869-77), it could be possible to express bispecific T cell engagers by the tumor cells, where they would directly bind to the surface and expose a T cell binding site. If this is possible, such in situ expressed molecules could exert their action directly at the site of the tumor, overcoming many of aforementioned problems. Such an approach would also be advantageous with respect to other inherent properties of the adenoviral system used, in particular the shield design, and an increasing tissue specificity while reducing liver uptake.

Earlier reports also described BiTE's encoded in the genome of viruses. All known approaches are however limited to oncolytical viruses. EMBO Mol Med (2017) 9:1067-1087 utilizes the oncolytic virus Enadenotucirev to express an EpCAM/CD3-specific BiTE. Enadenotucirev is an oncolytic virus that is in clinical trials for various types of cancer. The same oncolytic virus is used in WO2018/041827 and WO2019/043020. Mol Ther (2013) 21:796-805 utilize a (helper-virus dependent) oncolytic virus expressing a CD44/CD3-specific BITE (CD44v6/CD3), a cytokine (IL-12) and a checkpoint inhibitor (anti-PD-L1 Ab). This system is used in combination with a HER2-CAR-T cell.

All systems disclosed so far make use of oncolytic viruses, thereby targeting the cancer cell directly. The use of a non-oncolytic virus has several advantages. First, the non-oncolytic virus used herein has a capacity of up to 37 kb. This is in strong contrast to the limited capacity of oncolytic adenoviruses which typically have a capacity of 3-4 kb. It is therefore possible to encode additional molecules into the non-oncolytic viruses of the present disclosure, such as cytokines and chemokines, which may further enhance the therapeutic activity. Second, there is an important safety aspect. It has been reported that under certain circumstances also healthy cells produce viral progeny after administration of oncolytic viruses to patients. This is not the case with non-oncolytic viruses. Third, oncolytic viruses are more immunogenic than non-oncolytic viruses. This is due to the expression of viral proteins and the ensuing activation of the immune system. This may have the effect that the transduced cells are recognized and eliminated by the immune system which is particularly unfavorable when continuous expression of the therapeutic molecule is desired from other cell types than cancer cells.

Herein, we make use of an adapter system that enables us to direct the adenovirus expressing the bispecific T cell engagers to any cell type of interest, in particular cells of the tumor micro environment, thereby providing an increased flexibility for therapeutic interventions. The non-oncolytic viruses are non-replicating, and are not altered or designed to directly kill to the target cells. Instead, the non-oncolytic viruses are rather engineered to express the bispecific T cell engagers, at or in the proximity of the target site, e.g. a disease site in a human patient. This was achieved by way of a sophisticated molecular architecture of the polypeptides which were added to the non-oncolytic virus and thus bind to the capsid, thereby allowing retargeting to the cell type of interest. The therapeutic effect induced by the BiTE can be complemented with additional elements, e.g. by arming the adenovirus with additional components encoded on its genome, such as cytokines or additional moieties with a dedicated function and/or specificity, depending on the specific case and the target cell.

### SUMMARY OF THE INVENTION

In certain embodiments the present disclosure relates to a recombinant non-oncolytic virus comprising a bispecific T cell engager and a recombinant adapter molecule. In certain embodiments said bispecific T cell engager comprises
a) a first binding domain comprising a VH domain and a VL domain that bind to a T cell surface antigen, and
b) a second binding domain comprising a designed ankyrin repeat domain which binds to a target antigen exposed on the cell surface. In certain embodiments said T cell surface antigen is CD3.

In certain embodiments of the present disclosure, said VH domain of said first binding domain is covalently linked to said VL domain of said first binding domain by a first linker of sufficient length such that said VH domain and said VL domain fold to form a first binding domain that binds to said T cell surface antigen.

In certain embodiments of the present disclosure, said first binding domain and said second binding domain are covalently linked by a second linker of sufficient length such that said first binding domain and said second binding domain fold independently of each other.

In certain embodiments of the present disclosure, said non-oncolytic virus is an adenovirus. In certain embodiments said adenovirus is of adenovirus serotype 5 or comprises a knob of an adenovirus of serotype 5. In certain embodiments of the present disclosure, said adenovirus is a gutless, a shielded or a helper-dependent adenovirus.

In certain embodiments of the present disclosure, said bispecific T cell engager is encoded in the genome of the non-oncolytic virus.

In certain embodiments of the present disclosure, said non-oncolytic virus displays said recombinant adapter molecule.

In certain embodiments of the present disclosure, said recombinant adapter molecule comprises
a) a designed ankyrin repeat domain which binds to a target antigen exposed on the cell surface,
b) a designed ankyrin repeat domain which binds to the knob of the adenovirus, and
c) a trimerization domain. In certain embodiments, said trimerization domain is or is derived from the capsid protein SHP of lambdoid phage 21. In certain embodiments, said trimerization domain comprises the amino acid sequence of SEQ ID No. 1.

In certain embodiments of the present disclosure, said designed ankyrin repeat domain that binds to a knob of an adenovirus comprises the amino acid sequence of SEQ ID No. 2.

In certain embodiments of the present disclosure, said recombinant adapter molecule comprises from the N- to the C-terminus
a) said designed ankyrin repeat domain which binds to a target antigen exposed on the cell surface,
b) said designed ankyrin repeat domain which binds to the knob of the adenovirus, and
c) said trimerization domain.

In certain embodiments of the present disclosure, said first binding domain of said bispecific protein comprises a HCDR1 of SEQ ID No. 3, a HCDR2 of SEQ ID No. 4, a HCDR3 of SEQ ID No. 5, a LCDR1 of SEQ ID No. 6, a LCDR2 of SEQ ID No. 7 and a LCDR3 of SEQ ID No. 8.

In certain embodiments of the present disclosure, said first linker is a glycine-serine linker. In certain embodiments of the present disclosure, said second linker is a glycine-serine linker.

In certain embodiments of the present disclosure, said target antigen bound by said second binding domain of said bispecific T cell engager and said target antigen exposed on the cell surface and bound by the designed ankyrin repeat domain of said recombinant adapter molecule are the same target antigen. In certain embodiments said target antigen is HER2 (SEQ ID No. 12).

In certain embodiments of the present disclosure, said target antigen bound by said second binding domain of said bispecific T cell engager and said target antigen exposed on the cell surface and bound by the designed ankyrin repeat domain of said recombinant adapter molecule are different target antigens.

In certain embodiments of the present disclosure, said designed ankyrin repeat domain which binds to a target antigen exposed on the cell surface comprises SEQ ID No. 13.

In certain embodiments of the present disclosure, said non-oncolytic virus is for use in medicine. In certain embodiments said use in medicine is the use in the treatment of cancer.

In certain embodiments the present disclosure, provides a eukaryotic cell containing a non-oncolytic virus according to the present disclosure and/or a eukaryotic cell expressing a bispecific T cell engager encoded on the genome of a non-oncolytic virus.

### FIGURE LEGENDS

FIG. 1 shows the effect of the bispecific T cell engagers of the present disclosure on the metabolic activity of multiple HER2-positive cell lines with multiple donors. The bispecific T cell engagers lead to a dose dependent tumor killing.

FIG. 2 shows the IFNγ cytokine secretion of PBMCs upon contact with the bispecific T cell engager and cancer cell lines at depicted concentrations of FIG. 1.

FIG. 3 shows the IL-2 cytokine secretion of PBMCs upon contact with the bispecific T cell engager and cancer cell lines as depicted concentrations of FIG. 1.

FIG. 4 shows the effect of 200 nM purified BiTE E08-G3 on SKBR3 cells with and without the presence of PBMCs, cytotoxic activity was only observed in presence of both, E08-G3 and the effector cells.

FIG. 5 shows the expression of the bispecific T cell engagers by the target cells upon adenoviral delivery at various MOI's.

FIG. 6 shows the effect of the bispecific T cell engagers on the metabolic activity of target cells transduced with the non-oncolytic viruses encoding the bispecific T cell engagers of the present disclosure with and without the addition of PBMCs at various MOI's.

FIG. 7 shows that IL2 production of PBMCs mixed with a cancer cell line upon infecting the cancerous target cells with different MOIs of the non-oncolytic viruses of the present disclosures encoding the bispecific T cell engagers.

FIG. 8 shows that the metabolic activity in the target cell lines SKBR3 (top) and MCF7 (bottom) is decreased at a ratio of 1.2 and above (PBMCs per tumor cell) for the cell line SKBR3, and at a ratio of 0.6 and above for the cell line MCF7 upon infecting these cancerous cell lines with an MOI of 1 with non-oncolytic viruses encoding the bispecific T cell engagers.

FIG. 9 shows that the cell population treated with the non-oncolytic viruses of the present disclosure reduces the total amount of HER2 positive cells from around 26% down to about 6% of all cells. This effect takes only place if also PBMCs are present.

FIG. 10 shows that the cell population of FIG. 9 treated with non-oncolytic viruses of the present disclosure and PBMCs has about 20% less metabolic activity compared to the cell population treated with the non-oncolytic viruses alone.

FIG. 11 shows the experimental set-up of an in vivo experiment in a xenograft mice model

FIG. 12 shows that administration of virus in a xenograft mouse model resulted in reduction of tumor growth while control mice showed fast tumor progression.

FIG. 13 shows that mice treated with virus showed significantly longer survival compared to mice treated with T cells only. Statistical analysis was done with a Mantel-Cox test (****: p≤0.0001).

FIG. 14 shows that mice treated with adenovirally-delivered T cell engagers (DATE-AdV) showed a significant reduction in tumor growth as compared to recombinant DATEs (DATE protein), adenovirally-delivered GFP (GFP-AdV) and PBS.

FIG. 15 shows that 50% of mice treated with adenovirally-delivered DATEs went into complete remission and remained tumor free for 91 days.

FIG. 16 shows that treatment with adenovirally-delivered DATEs resulted in extended survival indicating prolonged expression of adenovirally-delivered DATEs and improved efficacy by continuous expression (FIG. 16).

FIG. 17 shows a qPCR analysis confirming successful transduction of cells at the tumor site.

FIG. 18 shows that significant delay in tumor growth was also observed upon i.v. injection of adenovirally-delivered DATEs.

FIG. 19 shows increased proinflammatory TNFα concentrations upon i.v. injection of adenovirally-delivered DATEs.

### DEFINITIONS

The term “recombinant” as used in recombinant protein, recombinant protein domain, recombinant non-oncolytic virus, recombinant adapter molecule and the like, means that said polypeptides or proteins, or said polypeptides or proteins comprised in said non-oncolytic virus, are produced by the use of recombinant DNA technologies well known by the practitioner skilled in the relevant art. For example, a recombinant DNA molecule (e.g. produced by gene synthesis) encoding a polypeptide can be cloned into a bacterial expression plasmid (e.g. pQE30, Qiagen). When such a constructed recombinant expression plasmid is inserted into a host cell (e.g. E. coli), this host cell can produce the polypeptide encoded by this recombinant DNA. The correspondingly produced polypeptide is called a recombinant polypeptide or recombinant protein. The non-oncolytic virus comprising such recombinant polypeptide or recombinant protein is called recombinant non-oncolytic virus.

The term “protein” as used herein refers to a polypeptide, wherein at least part of the polypeptide has, or is able to, acquire a defined three-dimensional arrangement by forming secondary, tertiary, or quaternary structures within and/or between its polypeptide chain(s). If a protein comprises two or more polypeptides, the individual polypeptide chains may be linked non-covalently or covalently, e.g. by a disulfide bond between two polypeptides.

A part of a protein, which individually has, or is able to acquire a defined three-dimensional arrangement by forming secondary or tertiary structures, is termed “protein domain” or “domain”. Such protein domains are well known to the practitioner skilled in the art.

The term “polypeptide” as used herein refers to a molecule consisting of one or more chains of multiple, i.e. two or more, amino acids linked via peptide bonds. A polypeptide typically consists of more than twenty amino acids linked via peptide bonds.

The term “peptide” as used herein refers to as used herein refers to a molecule consisting of one or more chains of multiple, i.e. two or more, amino acids linked via peptide bonds. A peptide typically consists of not more than twenty amino acids linked via peptide bonds.

The terms “designed ankyrin repeat protein”, “designed ankyrin repeat domain” and “DARPin” as used herein refer artificial polypeptides, comprising several ankyrin repeat motifs. These ankyrin repeat motifs provide a rigid interface arising from typically three repeated β-turns. DARPins usually carry two three repeats corresponding to an artificial consensus sequence, wherein six positions per repeat are randomized, flanked by two capping repeats with a hydrophilic surface (Curr Olpin Chem Biol (2009) 13:245-55; WO 02/20565).

The term “antibody” as used herein refers to a protein comprising at least two heavy (H) chains and two light (L) chains inter-connected by disulfide bonds, which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The term “antibody fragment” as used herein refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as “single chain antibody”, “single chain variable fragment”, “single chain Fv” or “scFv”; see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1 126-1 136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

The term “immunoglobulin” as used herein refers to any polypeptide or fragment thereof from the class of polypeptides known to the skilled person under this designation and comprising at least one antigen binding site. Preferably, the immunoglobulin is a soluble immunoglobulin from any of the classes IgA, IgD, IgE, IgG, or IgM, or a fragment comprising at least one antigen binding site derived thereof. Also comprised as immunoglobulins of the present invention are a bispecific immunoglobulin, a synthetic immunoglobulin, an immunoglobulin fragment, such as Fab, Fv or scFv fragments etc., a single chain immunoglobulin, and a nanobody. Further included are chemically modified derivatives of any of the aforesaid, e.g. PEGylated derivatives, as well as fusion proteins comprising any of the aforesaid immunoglobulins and fragments thereof. The immunoglobulin may be a human or humanized immunoglobulin, a primatized, or a chimerized immunoglobulin or a fragment thereof as specified above. Preferably, the immunoglobulin of the present invention is a polyclonal or a monoclonal immunoglobulin, more preferably a monoclonal immunoglobulin or a fragment thereof as specified above.

The terms “binds”, “is specific” and “specifically binds” as used herein refers to a molecule, for example an antibody or an antibody fragment, which recognizes a specific antigen, but does not substantially recognize or bind other molecules in a sample. An antibody that specifically binds to an antigen from one species may also bind to that antigen from one or more further species. Such cross-species reactivity does not itself alter the classification of an antibody as specific. In this context, the term “binding domain” refers to the domain of a protein or a polypeptide which is responsible for binding to a specific molecule or other protein or polypeptide.

The term “bispecific” as used herein refers to a molecule, for example an antibody or a polypeptide, which specifically binds two different antigens or to two different epitopes on the same antigen. The bispecific T cell engagers of the present disclosure are exemplary bispecific molecule.

The term “bispecific T cell engager” or “BiTE” as used herein refers to a bispecific polypeptide comprising two binding domains, wherein the first binding domain is specific for a T cell surface antigen and the second binding domain is specific for a target antigen exposed on the cell surface. The second binding domain may be any surface antigen of any cell. Preferred cells are diseased cells, such as malignant cell, cancerous cells or cell of the tumor micro environment. The first binding domain is specific for a T cell surface antigen, particularly a cytotoxic T cell. The most commonly used T cell surface antigen is CD3, but any other T cell surface antigen may be targeted as well, such as CD27, CD28, CD30, 4-1BB, OX40, ICOS (aka CD134) or GITR.

The term “epitope” refers to a site on an antigen to which a binding molecule or binding domain, such as an antibody, a single chain antibody or a designed ankyrin repeat domain specifically binds. Epitopes can be formed both from contiguous amino acids or non-contiguous amino acids juxtaposed by tertiary folding of a protein.

The term “nucleic acid” as used herein refers to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “vector” as used herein means a construct, which is capable of delivering, and usually expressing or regulating expression of, one or more gene(s) or nucleic acid(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “host cell” as used herein refers to any kind of cellular system which can be engineered to generate molecules according to the present disclosure. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Host cells according to the present disclosure can be a “eukaryotic cell” and include yeast and mammalian cells, including murine cells and from other rodents, preferably vertebrate cells such as those from a mouse, rat, monkey or human cell line, for example HKB11 cells, PERC.6 cells, HEL293T cells, CHO cells or any type of HEK cells, such as HEK293 cells or HEK 993 cells. Also suspension cell lines like CHO-S or HEK993 cells, or insect cell cultures like Sf9 cells may be used.

Host cells according to the present disclosure can also be “procaryotic cell” and include bacterial cells, such Escherichia coli. Certain strains of Escherichia coli may be particularly useful for expression of the molecules of the present disclosure, such as Escherichia coli strain DH5 (available from Bethesda Research Laboratories, Inc., Bethesda, Md/US).

The recombinant adapter molecules of the present disclosure form trimers that are highly stable. Each monomer contains a domain responsible for the formation of trimers which is referred to herein as “trimerization domain”. A preferred trimerization domain is the capsid protein SHP of lambdoid phage 21 (J Mol Biol; 344 (1): 179-93; PNAS 110 (10): E869-77 (2013)). SHP of lambdoid phage 21 has the following amino acid sequence:

(SEQ ID No. 1)
VRIFAGNDPAHTATGSSGISSPTPALTPLMLDEATGKLVVWDGQKAGSA
VGILVLPLEGTETALTYYKSGTFATEAIHWPESVDEHKKANAFAGSALS
HAALP

The term “stable trimer”, “trimeric protein” or “trimeric adapter” as used herein refers to a protein trimer by protein monomers comprising a trimerization domain, and wherein said trimer exhibits a stability which is higher than other, conventional protein trimers. For example, a stable trimer has a higher functional stability, a higher kinetic stability, or a higher high life for unfolding than other protein trimers. An example of a stable trimer is a trimer formed by monomers comprising the trimerization domain of the capsid protein SHP of lambdoid phage 21.

The term “derived from” in the context of an amino acid sequence refers to an amino acid sequence that is different to an original amino acid sequence, but maintains the function or activity of the original amino acid sequence.

The term “adenovirus” as used herein refers to any adenovirus, i.e. to human and non-human serotypes. The human isolates are classified into subgroups A-G. A preferred adenovirus of the present disclosure is adenovirus subtype 5 (“HadV-C5”). HadV-C5 includes modified version of the virus, such as replication-deficient HadV-C5 version, e.g. containing an E1/E3 deletion and/or one or more of the 4 mutations in the HVR7 (I421G, T423N, E424S and L426Y) (Nat. Commun. 9, 450 (2018)).

The terms “CAR” and “CXADR” as used herein refers to coxsackievirus and adenovirus receptor (UniProt: P78310). CAR is a type I membrane receptor for coxsackie viruses and adenoviruses.

The term “gutless” as used herein refers to an adenovirus that has been deleted of all viral coding regions.

The term “shielded” as used herein refers to an adenovirus which carries a shield, to protect the virion from undesired host interactions. Shielding can be achieved by various means, for example by using hexon-specific scFv's, such as 9C12 (Nature Communication (2018) 9:450).

The term “knob” as used herein refers to a knob on the end of the adenovirus fiber (e.g. GenBank: AAP31231.1) that binds to the cellular receptor. The knob of adenovirus subtype 5 binds to CAR. Some adenoviruses carry mutations in the gene encoding the knob protein. Adenoviruses having a four-amino acid deletion within the FG loop of the knob (TAYT mutation) show a decreased ability of the mutated knob to bind to CAR (Science, 286:1568-1571 (1999); J Mol Biol 405 (2): 410-426). Adenoviruses carrying four amino acid mutations in the hypervariable region 7 (HVR7 mutation) show a strongly reduced binding to blood coagulation factor X (Nat Commun (2018) 9:450).

The molecules of the present invention contain a designed ankyrin repeat domain that binds to the knob of an adenovirus. A preferred designed ankyrin repeat domain that binds to a knob is DARPin 1D3. Another preferred designed ankyrin repeat domain that binds to a knob is DARPin 1D3nc, a derivative of 1D3nc containing a stabilized C-cap. DARPin 1D3 has the following amino acid sequence:

(SEQ ID No. 2)
RSDLGKKLLEAARAGQDDEVRILMANGADANAYDHYGRTPLHMAAAVGH
LEIVEVLLRNGADVNAVDTNGTTPLHLAASLGHLEIVEVLLKYGADVNA
KDATGITPLYLAAYWGHLEIVEVLLKHGADVNAQDKFGKTPFDLAIDNG
NEDIAEVLQG

The term “CD3” as used herein refers to human CD3 (cluster of differentiation 3), a protein complex and T cell co-receptor that involved in activating both the cytotoxic T cell (CD8+ naive T cells) and T helper cells (CD4+ naive T cells). It is composed of four distinct chains. The complex contains a CD3γ chain, a CD38 chain, and two CD38 chains. CD3 is expressed on T cells in association with the T cell receptor complex (TCR) and is required for T cell activation. Antibodies binding to CD3 have been shown to cluster CD3 on T cells, thereby causing T cell activation in a manner similar to the engagement of the T-Cell receptor (TCR) by peptide-loaded MHC molecules. Bi- or multispecific antibody formats that co-engage CD3 and one or more cancer associated antigens have been developed to redirect T-cells to attack and lyse cancer cells.

An “antigen-binding moiety which specifically binds to CD3” refers to any moiety, protein scaffold, such as an antibody or an antibody fragment, such as a single-chain Fv or a Fab fragment with binding specificity for CD3. Preferably, said antigen-binding moiety which specifically binds to CD3 bind to CD3E. In certain embodiments, the antigen-binding moiety which specifically binds to CD3 is a single-chain antibody. In other embodiments, the antigen-binding moiety which specifically binds to CD3 is a bispecific single-chain antibody. In certain embodiments, said antigen-binding moiety which specifically binds to CD3 comprises a HCDR1 of TYAMN (SEQ ID No. 3), a HCDR2 of RIRSKYNNYATYYADSVKD (SEQ ID No. 4), a HCDR3 of HGNFGNSYVSWFAY (SEQ ID No. 5), a LCDR1 of RSSTGAVTTSNYAN (SEQ ID No. 6), a LCDR2 of GTNKRAP (SEQ ID No. 7) and a LCDR3 of ALWYSNLWV (SEQ ID No. 8). In certain embodiments, said antigen-binding moiety which specifically binds to CD3 comprises a VH domain of

(SEQ ID No. 9)
EVQLVESGGGLVQPGGSLRLSCAASGFTESTYAMNWVRQAPGKGLEWVG
RIRSKYNNYATYYADSVKDRFTISRDDSKNSLYLQMNSLKTEDTAVYYC
VRHGNFGNSYVSWFAYWGQGTLVTVSS,

and a VL domain of

(SEQ ID No. 10)
QAVVTQEPSLTVSPGGTVTLTCRSSTGAVTTSNYANWVQQKPGQAPRGL
IGGTNKRAPWTPARFSGSLLGGKAALTITGAQAEDEADYYCALWYSNLW
VFGGGTKLTVL.

CD3E (UniProt: P07766) has the following amino acid sequence:

(SEQ ID No. 11)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

The term “oncolytic virus” as used herein, refers to a virus which selectively infects, replicates in and kills tumor cells while having no or minimal effect on normal cells. Target cells are killed by cell lysis due to viral replication. Most therapeutically used oncolytic viruses are genetically engineered, for example for tumor selectivity, although some naturally occurring oncolytic viruses do exits, such as reovirus or senecavirus, that have been tested in clinical trials.

In contrast, the term “non-oncolytic virus” as used herein, refers to a virus that does not replicate in tumor cells. A non-oncolytic virus does not infect and kill tumor cells directly, but exerts its mechanism of action indirectly, for example, as in the present disclosure, via secretion of a bispecific single chain antibody which directs T cells to the cancerous site.

The term “non-replicating” as used herein, refers to a virus which lacks the ability to replicate following infection of a target cell.

The term “displaying” as used herein refers to the presentation of a polypeptide on the outside of an entity, such as an adenovirus or a non-oncolytic virus. The polypeptides so presented on the entity may be covalently or non-covalently attached to such entity. In the context of the present disclosure adapter molecules are recombinantly expressed and displayed on an adenoviruses or a non-oncolytic virus. This can be accomplished via a binding moiety or a scaffold, such as a designed ankyrin repeat domain that binds to the knob of an adenovirus. Alternatively, moiety or scaffold can also be genetically fused to an adenoviral protein, such as the hexon.

The term “HER2” as used herein, refers a member of the epidermal growth factor (EGF) receptor family of receptor tyrosine kinases. HER2 is also known as ErbB2. HER2 (UniProt: P04626) has the following amino acid sequence:

(SEQ ID No. 12)
MELAALCRWGLLLALLPPGAASTQVCTGTDMKLRLPASPETHLDMLRHL
YQGCQVVQGNLELTYLPTNASLSFLQDIQEVQGYVLIAHNQVRQVPLQR
LRIVRGTQLFEDNYALAVLDNGDPLNNTTPVTGASPGGLRELQLRSLTE
ILKGGVLIQRNPQLCYQDTILWKDIFHKNNQLALTLIDTNRSRACHPCS
PMCKGSRCWGESSEDCQSLTRTVCAGGCARCKGPLPTDCCHEQCAAGCT
GPKHSDCLACLHFNHSGICELHCPALVTYNTDTFESMPNPEGRYTFGAS
CVTACPYNYLSTDVGSCTLVCPLHNQEVTAEDGTQRCEKCSKPCARVCY
GLGMEHLREVRAVTSANIQEFAGCKKIFGSLAFLPESFDGDPASNTAPL
QPEQLQVFETLEEITGYLYISAWPDSLPDLSVFQNLQVIRGRILHNGAY
SLTLQGLGISWLGLRSLRELGSGLALIHHNTHLCFVHTVPWDQLFRNPH
QALLHTANRPEDECVGEGLACHQLCARGHCWGPGPTQCVNCSQFLRGQE
CVEECRVLQGLPREYVNARHCLPCHPECQPQNGSVTCFGPEADQCVACA
HYKDPPFCVARCPSGVKPDLSYMPIWKFPDEEGACQPCPINCTHSCVDL
DDKGCPAEQRASPLTSIISAVVGILLVVVLGVVFGILIKRRQQKIRKYT
MRRLLQETELVEPLTPSGAMPNQAQMRILKETELRKVKVLGSGAFGTVY
KGIWIPDGENVKIPVAIKVLRENTSPKANKEILDEAYVMAGVGSPYVSR
LLGICLTSTVQLVTQLMPYGCLLDHVRENRGRLGSQDLLNWCMQIAKGM
SYLEDVRLVHRDLAARNVLVKSPNHVKITDFGLARLLDIDETEYHADGG
KVPIKWMALESILRRRFTHQSDVWSYGVTVWELMTFGAKPYDGIPAREI
PDLLEKGERLPQPPICTIDVYMIMVKCWMIDSECRPRFRELVSEFSRMA
RDPQRFVVIQNEDLGPASPLDSTFYRSLLEDDDMGDLVDAEEYLVPQQG
FFCPDPAPGAGGMVHHRHRSSSTRSGGGDLTLGLEPSEEEAPRSPLAPS
EGAGSDVFDGDLGMGAAKGLQSLPTHDPSPLQRYSEDPTVPLPSETDGY
VAPLTCSPQPEYVNQPDVRPQPPSPREGPLPAARPAGATLERPKTLSPG
KNGVVKDVFAFGGAVENPEYLTPQGGAAPQPHPPPAFSPAFDNLYYWDQ
DPPERGAPPSTFKGTPTAENPEYLGLDVPV.

In certain embodiments, the binding moiety which binds to an epitope of a target antigen exposed on the cell surface is a designed ankyrin repeat domain. In certain embodiments, said designed ankyrin repeat domain which binds to an epitope of a target antigen exposed on the cell surface comprises the amino acid sequence

(SEQ ID No. 13)
DLGKKLLEAARAGQDDEVRILMANGADVNAKDEYGLTPLYLATAHGHLE
IVEVLLKNGADVNAVDAIGFTPLHLAAFIGHLEIAEVLLKHGADVNAQD
KFGKTAFDISIGNGNEDLAEILQ.

The term “linker” as used herein refers a molecule or macromolecule serving to connect different moieties or domains of a peptide or a polypeptide or, a protein/polypeptide domain and a non-protein/non-polypeptide moiety. Linkers can be of different nature. Different domains or modules within proteins are typically linked via peptide linkers. The term “flexible linker” as used herein refers to a peptide linker linking two different domains or modules of a protein and providing a certain degree of flexibility. Preferably, the flexible linker is hydrophilic and does not interacting with other surfaces. Commonly used flexible linkers are glycine-serine linkers (Biochemistry 56 (50): 6565-6574 (2017)). Glycine and serine are flexible and the adjacent protein domains are free to move relative to one another. Such flexible linkers are referred to herein as “glycine-serine linkers”. Other amino acids commonly used in respective linkers are proline, asparagine and threonine. Often the linker contains several repeats of a sequence of amino acids. A flexible linker used in the present disclosure is a (Gly₄Ser)₄-linker, i.e. a linker containing four repeats of the sequence glycine-glycine-glycine-glycine-serine. Other linkers that could be used in accordance with the present disclosure include but are not limited to PAS linkers, i.e. linkers containing repeats of the sequence proline-alanine-serine (Protein Eng Des Sel (2013) 26, 489-501 and charged linkers.

The term “short linker” as used herein refers to a peptide linker linking two different domains or modules of a protein and which is no longer than four, preferably no longer than three amino acids long. More preferably the short linker is no longer than two amino acids long. Alternatively the short linker is only one amino acid long. Alternatively the short linker is a single glycine residue.

The term “amino acid mutation” refers to amino acid substitutions, deletions, insertions, and modifications, as well as combinations thereof. Amino acid sequence deletions and insertions include N- and/or C-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful.

The term “variant” as used herein refers to a polypeptide that differs from a reference polypeptide by one or more amino acid mutation or modifications.

### EMBODIMENTS OF THE INVENTION

Disclosed herein is a system for the expression of bispecific T cell engagers in situ via non-oncolytic viruses. The system can be used to direct the viruses to any site of interest, including the tumor microenvironment. The system can be used in medicine, particularly in cancer-related disorders. Cargo, such as nucleic acids, in particular nucleic acids encoding therapeutically active or therapeutically helpful proteins and peptides, can be delivered to the target cells.

This is achieved by the present disclosure by making use of recombinant adapter molecules that are displayed on non-oncolytic viruses, such as adenoviruses, thereby targeting the viruses to the target cells, which then expresses the bispecific T cell engagers encoded on the viral genome. The system is functional with adenoviruses of any kind, i.e. first-generation virus, as well as high-capacity, helper virus-dependent adenoviral systems. The system is also functional with shielded adenoviruses. The system is also functional with other viruses, e.g. viruses that are engineered to carry a knob of an adenovirus of subtype 5.

### Non-Oncolytic Virus

The present disclosure makes use of a non-oncolytic virus, i.e. a virus that does not replicate in and kill tumor cells directly. Therefore, in certain embodiments the present disclosure relates to a non-oncolytic virus comprising a bispecific T cell engager, wherein said bispecific T cell engager comprises
a) a first binding domain comprising a VH domain and a VL domain that bind to a T cell surface antigen, and
b) a second binding domain comprising a designed ankyrin repeat domain which binds to a target antigen exposed on the cell surface.

In certain embodiments said VH domain is covalently linked to said VL domain by a first linker of sufficient length such that said VH domain and said VL domain fold to form a first binding domain that binds to said T cell surface antigen.

In certain embodiments, said T cell surface antigen is CD3. Therefore, in certain embodiments the present disclosure relates to a non-oncolytic virus comprising a bispecific T cell engager, wherein said bispecific T cell engager comprises
a) a first binding domain comprising a VH domain and a VL domain that bind to CD3, and
b) a second binding domain comprising a designed ankyrin repeat domain which binds to a target antigen exposed on the cell surface. In certain embodiments said VH domain is covalently linked to said VL domain by a first linker of sufficient length such that said VH domain and said VL domain fold to form a first binding domain that binds to CD3.

An exemplary non-oncolytic virus that can be used in the context of the present disclosure is an adenovirus, such as adenovirus subtype 5. It will be understood that also other adenoviral serotypes may be used in the spirit of the present disclosure, including human adenovirus serotype c5 (hAdV-C5), hAdV2, hAdV3, hAdV-B35, hAdV-D26, as well as hybrids thereof. A list of adenoviruses can be found on the website of the Human Adenovirus Working group (http://hadvwg.gmu.edu). Also, non-human adenoviruses may be used within the scope of the present disclosure, such as the AstraZeneca vaccine chimpanzee adenovirus Y25 (CHAdY25), or non-human adenoviral vectors were developed from bovine (Bad), canine (Cad), chimpanzee (ChAd), ovine (Oad), porcine (Pad), or fowl (Fad).

Therefore, in certain embodiments the present disclosure relates to an adenovirus encoding a bispecific T cell engager, wherein said T cell engager comprises
a) a first binding domain comprising a VH domain and a VL domain that bind to a T cell surface antigen, and
b) a second binding domain comprising a designed ankyrin repeat domain which binds to an epitope of a target antigen exposed on the cell surface.

In certain embodiments said VH domain is covalently linked to said VL domain by a first linker of sufficient length such that said VH domain and said VL domain fold to form a first binding domain that binds to said T cell surface antigen.

In certain embodiments, said T cell surface antigen is CD3. Therefore, in certain embodiments the present disclosure relates to an adenovirus encoding a bispecific T cell engager, wherein said T cell engager comprises
a) a first binding domain comprising a VH domain and a VL domain that bind to CD3, and
b) a second binding domain comprising a designed ankyrin repeat domain which binds to an epitope of a target antigen exposed on the cell surface. In certain embodiments said VH domain is covalently linked to said VL domain by a first linker of sufficient length such that said VH domain and said VL domain fold to form a first binding domain that binds to CD3.

In preferred embodiments, said non-oncolytic virus is a non-replicating virus. In preferred embodiments, said adenovirus is an adenovirus of subtype 5.

The bispecific T cell engagers of the present disclosure are encoded on the genome of the non-oncolytic virus.

Therefore, in certain embodiments the present disclosure relates to a non-oncolytic virus encoding a bispecific T cell engagers, wherein said bispecific T cell engagers comprises
a) a first binding domain comprising a VH domain and a VL domain that bind to a T cell surface antigen, and
b) a second binding domain comprising a designed ankyrin repeat domain which binds to an epitope of a target antigen exposed on the cell surface,
wherein said bispecific T cell engagers is encoded on the genome of the non-oncolytic virus. In certain embodiments, said T cell surface antigen is CD3.

In certain embodiments the present disclosure relates to an adenovirus expressing a bispecific T cell engagers, wherein said bispecific T cell engagers comprises
a) a first binding domain comprising a VH domain and a VL domain that bind to a T cell surface antigen, and
b) a second binding domain comprising a designed ankyrin repeat domain which binds to an epitope of a target antigen exposed on the cell surface,
wherein said bispecific T cell engagers is encoded on the genome of an adenovirus, and
wherein said adenovirus is a gutless adenovirus. In certain embodiments, said T cell surface antigen is CD3.

In certain embodiments the present disclosure relates to an adenovirus encoding a bispecific T cell engagers, wherein said bispecific T cell engagers comprises
a) a first binding domain comprising a VH domain and a VL domain that bind to a T cell surface antigen, and
b) a second binding domain comprising a designed ankyrin repeat domain which binds to an epitope of a target antigen exposed on the cell surface,
wherein said bispecific T cell engagers is encoded on the genome of an adenovirus, and
wherein said adenovirus is a shielded adenovirus. In certain embodiments, said T cell surface antigen is CD3.

### Bispecific T Cell Engagers

The bispecific T cell engagers of the present disclosure are encoded on the genome of the non-oncolytic virus. Therefore, in certain embodiments, the present disclosure relates to a recombinant non-oncolytic virus encoding a bispecific T cell engager in the genome.

In certain embodiments said non-oncolytic virus is an adenovirus. Therefore, in certain embodiments, the present disclosure relates to a recombinant adenovirus encoding a bispecific T cell engager in the genome The present disclosure relates to a recombinant non-oncolytic virus encoding a bispecific T cell engagers. In certain embodiments of the present disclosure, said bispecific T cell engager comprises
a) a first binding domain comprising a VH domain and a VL domain that bind to a T cell surface antigen, and
b) a second binding domain comprising a designed ankyrin repeat domain which binds to an epitope of a target antigen exposed on the cell surface. In certain embodiments, said T cell surface antigen is CD3.

In preferred embodiments, said non-oncolytic virus is a non-replicating virus

In certain embodiments, said VH domain is covalently linked to said VL domain by a first linker of sufficient length such that said VH domain and said VL domain fold to form a first binding domain that binds to CD3. In certain embodiments, said first binding domain and said second binding domain of said bispecific T cell engager are covalently linked by a second linker of a length such that said first binding domain and said second binding domain fold independently of each other. In preferred embodiments, said non-oncolytic virus is a non-replicating virus.

### Surface Molecules

In certain embodiments of the present disclosure, said bispecific T cell engagers comprise a binding domain comprising a designed ankyrin repeat domain which binds to an epitope of a target antigen exposed on the cell surface.

The designed ankyrin repeat domain of said bispecific T cell engager which binds to an epitope of a target antigen exposed on the cell surface and the designed ankyrin repeat domain, which is part of said recombinant adapter molecule and binds to an epitope of a target antigen exposed on the cell surface, may be identical.

The designed ankyrin repeat domain which binds to an epitope of a target antigen exposed on the cell surface of said bispecific T cell engager and the designed ankyrin repeat domain which binds to an epitope of a target antigen exposed on the cell surface of said recombinant adapter molecule may bind to the same target antigen, but to different epitopes of said target antigen.

The designed ankyrin repeat domain which binds to an epitope of a target antigen exposed on the cell surface of said bispecific T cell engager and the designed ankyrin repeat domain which binds to an epitope of a target antigen exposed on the cell surface of said recombinant adapter molecule may be different.

The designed ankyrin repeat domain which binds to an epitope of a target antigen exposed on the cell surface of said bispecific T cell engager and the designed ankyrin repeat domain which binds to an epitope of a target antigen exposed on the cell surface of said recombinant adapter molecule may bind to different target antigens.

Said target antigen exposed on the cell surface can be any antigen which is at least partially exposed on a cell, so that the respective epitope can be recognized and bound by said binding domain. In most cases, such a molecule will be located in or on the plasma membrane of the cell such that at least part of this molecule remains accessible from outside the cell in tertiary form, i.e. its correctly folded native structure. A non-limiting example of a cell surface molecule, which is located in the plasma membrane is a transmembrane protein comprising, in its tertiary conformation, regions of hydrophilicity and hydrophobicity. Here, at least one hydrophobic region allows the cell surface molecule to be embedded, or inserted in the hydrophobic plasma membrane of the cell while the hydrophilic regions extend on either side of the plasma membrane into the cytoplasm and extracellular space, respectively. A non-limiting list of possible surface antigen includes avβ36 integrin, BCMA, B7-H3, B7-H4, B7-H6, carbonic anhydrase 9, CTAG, CEA, a cyclin (e.g. cyclin A2), CCL-1, CD19, CD20, CD22, CD23, CD24, CD30, CD33, CD38, CD44, CD44v6, CD44v7/8, CD45, CD123, CD133, CD138, CD171, CSPG4, EGFR, EPG-2, EPG-40, ephrinB2, ephrin receptor A2, estrogen receptor, FCRL5, fetal AchR, a folate binding protein (FBP), Flt3, folate receptor alpha, ganglioside GD2, OGD2, ganglioside GD3, gp100 100, GPC3, GPRC5D, EGFR, Her2, Her3, Her4, erbB dimers, HMW-MAA), EpCAM, hepatitis B surface antigen, HLA-A1, HLA-A2, IL-22 receptor alpha, IL-13 receptor alpha 2, kappa light chain, LI-CAM), LRRC8A, MAGE, MAGE-A3, MAGE-A6, MAGE-A10, mesothelin, c-Met, MUC1, MUC16, NKG2D ligands, MART-1, NCAM), oncofetal antigen, PRAME, progesterone receptor, PSCA, PSMA, ROR1, ROR2, survivin, TPBG, TAG72, TRP1, TRP2, DCT), VEGFR and, VEGFR2.

Therefore, in certain embodiments, the present disclosure relates to a recombinant non-oncolytic virus encoding a bispecific T cell engager, wherein said bispecific single chain antibody comprises
a) a first binding domain comprising a VH domain and a VL domain that bind to CD3, and
b) a second binding domain comprising a designed ankyrin repeat domain which binds to an epitope of a target antigen exposed on the cell surface,
wherein said target antigen exposed on the cell surface is selected from the group of avβ36 integrin, BCMA, B7-H3, B7-H4, B7-H6, carbonic anhydrase 9, CTAG, CEA, a cyclin (e.g. cyclin A2), CCL-1, CD19, CD20, CD22, CD23, CD24, CD30, CD33, CD38, CD44, CD44v6, CD44v7/8, CD45, CD123, CD133, CD138, CD171, CSPG4, EGFR, EPG-2, EPG-40, ephrinB2, ephrin receptor A2, estrogen receptor, FCRL5, fetal AchR, a folate binding protein (FBP), Flt3, folate receptor alpha, ganglioside GD2, OGD2, ganglioside GD3, gp100 100, GPC3, GPRC5D, EGFR, Her2, Her3, Her4, erbB dimers, HMW-MAA), EpCAM, hepatitis B surface antigen, HLA-A1, HLA-A2, IL-22 receptor alpha, IL-13 receptor alpha 2, kappa light chain, LI-CAM), LRRC8A, MAGE, MAGE-A3, MAGE-A6, MAGE-A10, mesothelin, c-Met, MUC1, MUC16, NKG2D ligands, MART-1, NCAM), oncofetal antigen, PRAME, progesterone receptor, PSCA, PSMA, ROR1, ROR2, survivin, TPBG, TAG72, TRP1, TRP2, DCT), VEGFR and VEGFR2.

In other embodiments, the present disclosure relates to a recombinant non-oncolytic virus comprising a recombinant adapter molecule comprising a designed ankyrin repeat domain which binds to an epitope of a target antigen exposed on the cell surface,
wherein said target antigen exposed on the cell surface is selected from the group of avβ6 integrin, BCMA, B7-H3, B7-H4, B7-H6, carbonic anhydrase 9, CTAG, CEA, a cyclin (e.g. cyclin A2), CCL-1, CD19, CD20, CD22, CD23, CD24, CD30, CD33, CD38, CD44, CD44v6, CD44v7/8, CD45, CD123, CD133, CD138, CD171, CSPG4, EGFR, EGFR, EPG-2, EPG-40, ephrinB2, ephrin receptor A2, estrogen receptor, FCRL5, fetal AchR, a folate binding protein (FBP), Flt3, folate receptor alpha, ganglioside GD2, OGD2, ganglioside GD3, gp100 100, GPC3, GPRC5D, Her2, Her3, Her4, erbB dimers, HMW-MAA), EpCAM, hepatitis B surface antigen, HLA-A1, HLA-A2, IL-22 receptor alpha, IL-13 receptor alpha 2, kappa light chain, LI-CAM), LRRC8A, MAGE, MAGE-A3, MAGE-A6, MAGE-A10, mesothelin, c-Met, MUC1, MUC16, NKG2D ligands, MART-1, NCAM), oncofetal antigen, PRAME, progesterone receptor, PSCA, PSMA, ROR1, ROR2, survivin, TPBG, TAG72, TRP1, TRP2, DCT), VEGFR and VEGFR2.

In certain embodiments, the surface antigen is HER2. In other embodiments, the surface antigen comprises the amino acid sequence of SEQ ID No. 12.

Therefore, in certain embodiments the present disclosure relates to a non-oncolytic virus encoding a bispecific T cell engager, wherein said bispecific T cell engager comprises
a) a first binding domain that binds to a T cell surface antigen, and
b) a second binding domain comprising a designed ankyrin repeat domain that binds to HER2. In certain embodiments, said T cell surface antigen is CD3.

In other embodiments, the present disclosure relates to a recombinant non-oncolytic virus encoding a recombinant adapter molecule comprising a designed ankyrin repeat domain which binds to HER2.

In other embodiments the present disclosure relates to a non-oncolytic virus encoding a bispecific single chain antibody, wherein said bispecific single chain antibody comprises
a) a first binding domain that binds to a T cell surface antigen, and
b) a second binding domain comprising a designed ankyrin repeat domain that binds to a polypeptide comprising the amino acid sequence of SEQ ID No. 12. In certain embodiments, said T cell surface antigen is CD3.

In other embodiments, the present disclosure relates to a recombinant non-oncolytic virus comprising a recombinant adapter molecule comprising a designed ankyrin repeat domain that binds to a polypeptide comprising the amino acid sequence of SEQ ID No. 12.

In certain embodiments of the present disclosure, the designed ankyrin repeat domain which binds to an epitope of a target antigen exposed on the cell surface comprises the amino acid sequence of SEQ ID No. 13. In other embodiments, the designed ankyrin repeat domain which binds to an epitope of a target antigen exposed on the cell surface is or is derived from the G3 DARPin. The G3 DARPin is described in Cancer Res (2010) 70:1595-1605.

Therefore, in certain embodiments the present disclosure relates to a non-oncolytic virus encoding a bispecific T cell engager, wherein said bispecific T cell engager comprises
a) a first binding domain that binds to a T cell surface antigen, and
b) a second binding domain comprising a designed ankyrin repeat domain comprising the amino acid sequence of SEQ ID No. 13. In certain embodiments, said T cell surface antigen is CD3.

In other embodiments, the present disclosure relates to a recombinant non-oncolytic virus comprising a recombinant adapter molecule comprising a designed ankyrin repeat domain comprising the amino acid sequence of SEQ ID No. 13.

In other embodiments the present disclosure relates to a non-oncolytic virus encoding a bispecific single chain antibody, wherein said bispecific single chain antibody comprises
a) a first binding domain that binds to a T cell surface antigen, and
b) a second binding domain which is or is derived from the G3 DARPin. In certain embodiments, said T cell surface antigen is CD3.

In other embodiments, the present disclosure relates to a recombinant non-oncolytic virus comprising a recombinant adapter molecule comprising a designed ankyrin repeat domain which is or is derived from the G3 DARPin.

### CD3

In certain embodiments of the present disclosure said bispecific T cell engager comprises a first binding domain comprising a VH domain and a VL domain that bind to CD3. In other embodiments of the present disclosure said bispecific T cell engager comprises a first binding domain comprising a VH domain and a VL domain that bind a polypeptide comprising the amino acid sequence of SEQ ID No. 11.

Any known anti-CD3 antibody can be converted into a single chain antibody and be used within the spirit of the present disclosure, including the anti-CD3 antibodies disclosed in WO2004/108158, WO2005/118635, WO2007/033291, WO2007/042261, WO2008/119567, WO2012/158818, WO2012/162067, WO2016/020444, WO2015/001085, WO2014/047231, WO2014/129270, WO2014/110601, WO2015/095392, WO2015/063339, WO2016/036937, WO2016/204966, US2017/0157251, WO2017/136659, WO2018/117237, WO2019/034580, WO2019/078697, WO2020/114478, WO2022/063819 and WO2022/068809. As an exemplary anti-CD3 antibody, the CD3 arm of the bispecific anti-CD123/CD3 antibody flotetuzumab was used in the present disclosure. This anti-CD3 antibody is cross-reactive with CD3 from cynomolgus CD3 and rhesus CD3.

Therefore, in certain embodiments the present disclosure relates to a recombinant non-oncolytic virus encoding a bispecific T cell engager, wherein said bispecific T cell engager comprises
a) a first binding domain comprising a VH domain and a VL domain that bind to CD3, and
b) a second binding domain comprising a designed ankyrin repeat domain which binds to an epitope of a target antigen exposed on the cell surface,
wherein said first binding domain of said bispecific single chain antibody comprises a HCDR1 of SEQ ID No. 3, a HCDR2 of SEQ ID No. 4, a HCDR3 of SEQ ID No. 5, a LCDR1 of SEQ ID No. 6, a LCDR2 of SEQ ID No. 7 and a LCDR3 of SEQ ID No. 8.

In other embodiments the present disclosure relates to a recombinant non-oncolytic virus encoding a bispecific T cell engager, wherein said bispecific T cell engager comprises
a) a first binding domain comprising a VH domain and a VL domain that bind to CD3, and
b) a second binding domain comprising a designed ankyrin repeat domain which binds to an epitope of a target antigen exposed on the cell surface,
wherein said first binding domain of said bispecific T cell engager comprises the VH domain of SEQ ID No. 9 and the VL domain of SEQ ID No. 10. In other embodiments, said first binding domain specifically binding to CD3 comprises an amino acid sequence with at least 90%, preferably at least 95% and more preferably at least 98% homology to the VH domain of SEQ ID No. 9 and/or the VL domain of SEQ ID No. 10.

In certain embodiments, said first binding domain specifically binding to CD3 competes for binding to CD3 with an antigen-binding moiety comprising a HCDR1 of SEQ ID No. 3, a HCDR2 of SEQ ID No. 4, a HCDR3 of SEQ ID No. 5, a LCDR1 of SEQ ID No. 6, a LCDR2 of SEQ ID No. 7 and a LCDR3 of SEQ ID No. 8.

Linker within the Bispecific Single Chain Antibody.

The first binding domain of the bispecific T cell engager comprises a first linker between the VH domain and the VL domain of said first binding domain. This first linker is of sufficient length, so that the VH domain and the VL domain can properly fold to form a functional first binding domain. Any commonly used linkers may be employed. Commonly used linkers are glycine-serine linker. A preferred glycine-serine linker is a (Gly₄Ser)₄-linker. A (Gly₄Ser)₄-linker has the following amino acid sequence:

(SEQ ID No. 14)
GGGGSGGGGSGGGGSGGGGS.

Therefore, in embodiments the present disclosure relates to a recombinant non-oncolytic virus encoding a bispecific T cell engager, wherein said bispecific T cell engager comprises
a) a first binding domain comprising a VH domain and a VL domain that bind to a T cell surface antigen, wherein said VH domain is covalently linked to said VL domain by a first linker of sufficient length such that said VH domain and said VL domain fold to form a first binding domain that binds to said T cell surface antigen, and
b) a second binding domain comprising a designed ankyrin repeat domain which binds to an epitope of a target antigen exposed on the cell surface,
wherein said first linker is a glycine serine linker. In certain embodiments, said T cell surface antigen is CD3.

In other embodiments the present disclosure relates to a recombinant non-oncolytic virus encoding a bispecific T cell engager, wherein said bispecific T cell engager comprises
a) a first binding domain comprising a VH domain and a VL domain that bind to a T cell surface antigen, wherein said VH domain is covalently linked to said VL domain by a first linker of sufficient length such that said VH domain and said VL domain fold to form a first binding domain that binds to said T cell surface antigen, and
b) a second binding domain comprising a designed ankyrin repeat domain which binds to an epitope of a target antigen exposed on the cell surface,
wherein said first linker is a (Gly₄Ser)₄ linker. In yet other embodiments, said linker consists of the amino acid sequence of SEQ ID No. 14. In certain embodiments, said T cell surface antigen is CD3.

The bispecific T cell engager also comprises a linker between the first binding domain and the second binding domain of said bispecific single chain antibody. This second linker is of sufficient length such that the first binding domain and the second binding domain fold independently of each other. Again, any commonly used linkers may be employed. Commonly used linkers are glycine-serine linker. A preferred glycine-serine linker is a (Gly₄Ser)₄-linker.

Therefore, in certain embodiments, the present disclosure relates to a non-oncolytic virus encoding a bispecific T cell engager comprising
a) a first binding domain comprising a VH domain and a VL domain that bind to a T cell surface antigen, wherein said VH domain is covalently linked to said VL domain by a first linker of sufficient length such that said VH domain and said VL domain fold to form a first binding domain that binds to said T cell surface antigen, and
b) a second binding domain comprising a designed ankyrin repeat domain which binds to an epitope of a target antigen exposed on the cell surface,
wherein said first binding domain and said second binding domain are covalently linked by a second linker of a length such that said first binding domain and said second binding domain fold independently of each other. In certain embodiments, said T cell surface antigen is CD3.

In other embodiments, the present disclosure relates to a non-oncolytic virus encoding a bispecific T cell engager comprising
a) a first binding domain comprising a VH domain and a VL domain that bind to a T cell surface antigen, and
b) a second binding domain comprising a designed ankyrin repeat domain which binds to an epitope of a target antigen exposed on the cell surface,
wherein said first binding domain and said second binding domain are covalently linked by a second linker of a length such that said first binding domain and said second binding domain fold independently of each other. In certain embodiments, said T cell surface antigen is CD3.

In other embodiments, the present disclosure relates to a non-oncolytic virus encoding a bispecific single chain antibody comprising
a) a first binding domain comprising a VH domain and a VL domain that bind to a T cell surface antigen, and
b) a second binding domain comprising a designed ankyrin repeat domain which binds to an epitope of a target antigen exposed on the cell surface,
wherein said first binding domain and said second binding domain are covalently linked by a second linker of a length such that said first binding domain and said second binding domain fold independently of each other, wherein said second linker is a glycine serine linker. In certain embodiments, said T cell surface antigen is CD3.

In other embodiments, the present disclosure relates to a non-oncolytic virus encoding a bispecific T cell engager comprising
a) a first binding domain comprising a VH domain and a VL domain that bind to a T cell surface antigen, and
b) a second binding domain comprising a designed ankyrin repeat domain which binds to an epitope of a target antigen exposed on the cell surface,
wherein said first binding domain and said second binding domain are covalently linked by a second linker of a length such that said first binding domain and said second binding domain fold independently of each other, wherein said second linker is a (Gly₄Ser)₄ linker. In yet other embodiments, said linker consists of the amino acid sequence of SEQ ID No. 14. In certain embodiments, said T cell surface antigen is CD3.

The anti-CD3 binding domain of the exemplified bispecific single chain antibody has the following sequence:

(SEQ ID No. 15)
EVQLVESGGGLVQPGGSLRLSCAASGFTFSTYAMNWVRQAPGKGLEWVG
RIRSKYNNYATYYADSVKDRFTISRDDSKNSLYLQMNSLKTEDTAVYYC
VRHGNFGNSYVSWFAYWGQGTLVTVSSGGGGSGGGGSGGGGSGGGGSQA
VVTQEPSLTVSPGGTVTLTCRSSTGAVTTSNYANWVQQKPGQAPRGLIG
GTNKRAPWTPARFSGSLLGGKAALTITGAQAEDEADYYCALWYSNLWVF
GGGTKLTVL.

Therefore, in certain embodiments, the present disclosure relates to a non-oncolytic virus encoding a bispecific T cell engager comprising
a) a first binding domain comprising a VH domain and a VL domain that bind to CD3 and wherein said first binding domain comprises the amino acid sequence of SEQ ID No. 15, and
b) a second binding domain comprising a designed ankyrin repeat domain which binds to an epitope of a target antigen exposed on the cell surface.

The full sequence of the exemplified T cell engager is as follows:

(SEQ ID No. 16)
GSEVQLVESGGGLVQPGGSLRLSCAASGFTFSTYAMNWVRQAPGKGLEW
VGRIRSKYNNYATYYADSVKDRFTISRDDSKNSLYLQMNSLKTEDTAVY
YCVRHGNFGNSYVSWFAYWGQGTLVTVSSGGGGSGGGGSGGGGSGGGGS
QAVVTQEPSLTVSPGGTVTLTCRSSTGAVTTSNYANWVQQKPGQAPRGL
IGGTNKRAPWTPARFSGSLLGGKAALTITGAQAEDEADYYCALWYSNLW
VFGGGTKLTVLGKLGGGGSGGGGSGGGGSGGGGSGRDLGKKLLEAARAG
QDDEVRILMANGADVNAKDEYGLTPLYLATAHGHLEIVEVLLKNGADVN
AVDAIGFTPLHLAAFIGHLEIAEVLLKHGADVNAQDKFGKTAFDISIGN
GNEDLAEILQ.

Therefore, in certain embodiments, the present disclosure relates to a non-oncolytic virus encoding a bispecific T cell engager comprising
a) a first binding domain comprising a VH domain and a VL domain that bind to CD3, and
b) a second binding domain comprising a designed ankyrin repeat domain which binds to HER2,
wherein said bispecific single chain antibody comprises the amino acid sequence of SEQ ID No. 16.

### Recombinant Adapter Molecules

In certain embodiments, the present disclosure makes use of recombinant adapter molecules comprising
a) a designed ankyrin repeat domain which binds to a target antigen exposed on the cell surface,
b) a designed ankyrin repeat domain which binds to the knob of the non-oncolytic virus, and
c) a trimerization domain. In certain embodiments, said non-oncolytic virus is an adenovirus.

Hence, in certain embodiments the present disclosure relates to a non-oncolytic virus, wherein said non-oncolytic virus comprises a recombinant adapter molecule comprising
a) a designed ankyrin repeat domain which binds to a target antigen exposed on the cell surface,
b) a designed ankyrin repeat domain which binds to the knob of a non-oncolytic virus, and
c) a trimerization domain. In certain embodiments, said non-oncolytic virus expresses and displays said recombinant adapted molecule. In certain embodiments, said non-oncolytic virus is an adenovirus.
   Ankyrin Repeat Domain which Binds to the Knob of the Non-Oncolytic Virus

In certain embodiments, the present disclosure makes use of recombinant adapter molecules comprising a designed ankyrin repeat domain which binds to the knob of a non-oncolytic virus, such as an adenovirus.

It will be appreciated that any designed ankyrin repeat domain with specificity for the knob of a non-oncolytic virus or adenovirus may be used within the spirit of the present disclosure. Exemplified herein is a designed ankyrin repeat domain derived from DARPin 1D3 (Proc. Natl. Acad. Sci. 110, E869-E877 (2013)). DARPin 1D3 binds to the knob of an adenovirus and comprises the amino acid sequence of SEQ ID No. 2. Used herein is 1D3nc, a derivative of 1D3 containing a stabilized C-cap.

It will also be understood that also variants of DARPin 1D3 may be used within the spirit of the present disclosure. In other words, the amino acid sequence of such modified DARPin 1D3 does not need to be identical to that of amino acid sequence of SEQ ID No. 2, but may contain amino acids mutations, provided that the function of DARPin 1D3, i.e. binding to the knob of an adenovirus is preserved. Also DARPins different than 1D3, but having the same target specificity, may be used within the scope of the present disclosure. Such new DARPin may for example be selected in a new screening campaign. Also binding entities different than DARPins, i.e. binders based on a different scaffold, but having the same target specificity as 1D3 might be used.

Therefore, in certain embodiments, the present disclosure relates to a recombinant adapter molecule comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said designed ankyrin repeat domain which binds to the knob of an adenovirus is DARPin 1D3.

In other embodiments, the present disclosure relates to a recombinant adapter molecule comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said designed ankyrin repeat domain which binds to the knob of an adenovirus is or is derived from DARPin 1D3.

In other embodiments, the present disclosure relates to a recombinant adapter molecule comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said designed ankyrin repeat domain which binds to the knob of an adenovirus is a variant of DARPin 1D3.

In other embodiments, the present disclosure relates to a recombinant adapter molecule comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said designed ankyrin repeat domain which binds to the knob of an adenovirus comprises the amino acid sequence of SEQ ID No. 2.

In other embodiments, the present disclosure relates to a recombinant adapter molecule comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said designed ankyrin repeat domain which binds to the knob of an adenovirus comprising a variant of the amino acid sequence of SEQ ID No. 2.

In other embodiments, the present disclosure relates to a non-oncolytic virus comprising any one of aforementioned recombinant adapter molecules. In other embodiments, the present disclosure relates to a non-oncolytic virus expressing and displaying any one of aforementioned recombinant adapter molecules. In other embodiments, the present disclosure relates to an adenovirus comprising any one of aforementioned recombinant adapter molecules. In other embodiments, the present disclosure relates to an adenovirus expressing and displaying any one of aforementioned recombinant adapter molecules.

The present disclosure can, however, also be practiced with other non-oncolytic viruses than adenoviruses. If another non-oncolytic virus is used a designed ankyrin repeat domain needs to be selected that binds to the knob of such non-oncolytic virus. Therefore, in certain embodiments the present disclosure relates to recombinant proteins comprising
a) a designed ankyrin repeat domain which binds to a target antigen exposed on the cell surface,
b) a designed ankyrin repeat domain which binds to the knob of the non-oncolytic virus, and
c) a trimerization domain.

Another possibility is to engineer the non-oncolytic virus in a so that the non-oncolytic virus carries the knob of an adenovirus of serotype 5. The recombinant protein disclosed herein, in particular recombinant protein comprising DARPin 1D3, may then be used with such non-oncolytic virus. Therefore, in certain embodiments the present disclosure relates to a non-oncolytic virus comprising:
a) a designed ankyrin repeat domain which binds to a target antigen exposed on the cell surface,
b) a designed ankyrin repeat domain which binds to the knob of an adenovirus, and
c) a trimerization domain,
wherein said designed ankyrin repeat domain binds to the knob of an adenovirus of subtype 5, and
wherein said non-oncolytic virus carries the knob of an adenovirus of serotype 5.

### Trimerization Domain

In certain embodiments, the present disclosure makes use of recombinant adapter molecules comprising a trimerization domain. The trimerization domain is responsible for the formation of trimers. Each monomer of the molecules of the present disclosure comprises a trimerization domain. Principally any trimerization domain may be used, provided it is stable and geometrically fits the knob of the non-oncolytic virus, e.g. the adenovirus, can be used. A preferred trimerization domain is the capsid protein SHP of lambdoid phage 21 (J Mol Biol; 344 (1): 179-93; PNAS 110 (10): E869-77 (2013)).

Therefore, in certain embodiments the present disclosure relates to recombinant adapter molecules comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said trimerization domain is the capsid protein SHP of lambdoid phage 21.

In other embodiments the present disclosure relates to recombinant adapter molecules comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said trimerization domain is derived from the capsid protein SHP of lambdoid phage 21.

In other embodiments the present disclosure relates to recombinant proteins comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said trimerization domain comprises the amino acid sequence of SEQ ID No. 1.

Also, other trimerization domains known to the skilled person may be used for the formation or trimers. Without being limited other potential trimerization domains include the trimerization domain involved in collagen folding (Int J Biochem Cell Biol 44:21-32 (2012)), the trimerization domain of T4 phage fibritin (PloS One 7: e43603 (2012) or the GCN4-based isoleucine zipper (J Biol Chem 290:7436-42 (2015)).

The trimerization domain is responsible for the formation of the trimeric adapter molecules. The trimers disclosed herein are extraordinary stable (J Mol Biol (2004) 344:179-93; PNAS (2013) 110 E869-77). In certain embodiments the trimeric adapter molecules of the present disclosure remain intact in SDS gel electrophoresis. In other embodiments the trimeric adapter molecules are not denatured in SDS gel electrophoresis. In other embodiments the trimeric adapter molecules have a half-life in solution of at least one week, preferably at least two week and even more preferably at least one month.

Therefore, in certain embodiments the present disclosure relates to recombinant adapter molecules comprising
a) a designed ankyrin repeat domain which binds to a target antigen exposed on the cell surface,
b) a designed ankyrin repeat domain which binds to the knob of the non-oncolytic virus, and
c) a trimerization domain,
wherein said trimerization domain has a half-life in solution of at least one week, preferably at least two weeks and even more preferably at least one month.

In certain embodiments, the present disclosure relates to a trimeric protein consisting of three recombinant adapter molecules as described herein above.

### Orientation

Principally, the individual parts of the recombinant adapter molecules of the present disclosure can be arranged in any order. In certain embodiments, the recombinant adapter molecule comprises from the N- to the C-terminus
a) said designed ankyrin repeat domain which binds a target antigen exposed on the cell surface,
b) said designed ankyrin repeat domain which binds to the knob of the adenovirus, and
c) said trimerization domain.

In certain embodiments, the present disclosure relates to a recombinant adapter molecule comprises from the N- to the C-terminus
a) a designed ankyrin repeat domain which binds to a target antigen exposed on the cell surface,
b) a designed ankyrin repeat domain which binds to the knob of the adenovirus comprising the amino acid sequence of SEQ ID No. 2, and
c) a trimerization domain.

In other embodiments, the present disclosure relates to a recombinant adapter molecule comprises from the N- to the C-terminus
a) a designed ankyrin repeat domain which binds to a target antigen exposed on the cell surface,
c) a designed ankyrin repeat domain which binds to the knob of the adenovirus, and
d) a trimerization domain comprising the amino acid sequence of SEQ ID No. 1.

In other embodiments, the present disclosure relates to a recombinant adapter molecule comprises from the N- to the C-terminus
a) a designed ankyrin repeat domain which binds to a target antigen exposed on the cell surface,
b) a designed ankyrin repeat domain which binds to the knob of the adenovirus comprising the amino acid sequence of SEQ ID No. 2, and
c) a trimerization domain comprising the amino acid sequence of SEQ ID No. 1.

In other embodiments, the present disclosure relates to a recombinant adapter molecule comprises from the N- to the C-terminus
a) a designed ankyrin repeat domain which binds to HER2,
b) a designed ankyrin repeat domain which binds to the knob of the adenovirus comprising the amino acid sequence of SEQ ID No. 2, and
c) a trimerization domain comprising the amino acid sequence of SEQ ID No. 1.

In other embodiments, the present disclosure relates to a recombinant adapter molecule comprises from the N- to the C-terminus
a) a designed ankyrin repeat comprising the amino acid sequence of SEQ ID No. 13,
b) a designed ankyrin repeat domain which binds to the knob of the adenovirus comprising the amino acid sequence of SEQ ID No. 2, and
c) a trimerization domain comprising the amino acid sequence of SEQ ID No. 1.

In alternative embodiments, the recombinant adapter molecule comprises from the N- to the C-terminus
a) a designed ankyrin repeat domain which binds to the knob of the adenovirus,
b) a trimerization domain, and
c) a designed ankyrin repeat domain which binds to a second epitope of a target antigen exposed on the cell surface.
   Linker within the Recombinant Adapter Molecule

The recombinant adapter molecules of the present disclosure may also comprise a flexible linker. If the recombinant adapter molecule comprises from the N- to the C-terminus
a) a designed ankyrin repeat domain which binds a target antigen exposed on the cell surface,
b) a designed ankyrin repeat domain which binds to the knob of the adenovirus, and
c) a trimerization domain,
then said flexible linker is between said designed ankyrin repeat domain which binds to a second epitope of a target antigen exposed on the cell surface and said designed ankyrin repeat domain which binds to the knob of the adenovirus.

Therefore, in certain embodiments the present disclosure relates to a recombinant adapter molecules comprising from the N- to the C-terminus
a) a designed ankyrin repeat domain which binds to a target antigen exposed on the cell surface,
b) a flexible linker,
c) a designed ankyrin repeat domain which binds to the knob of an adenovirus, and
d) a trimerization domain.

Principally any flexible linker can be used within the spirit of the present disclosure. Certain preferred flexible linkers are glycine-serine linkers. A particularly preferred flexible linker is a (Gly₄Ser)₄-linker.

Therefore, in certain embodiments the present disclosure relates to a recombinant adapter molecule comprising from the N- to the C-terminus
a) a designed ankyrin repeat domain which binds to a target antigen exposed on the cell surface,
b) a flexible linker,
c) a designed ankyrin repeat domain which binds to the knob of an adenovirus, and
d) a trimerization domain.
wherein said flexible linker is a glycine-serine linker.

In other embodiments the present disclosure relates to a recombinant adapter molecule comprising from the N- to the C-terminus
a) a designed ankyrin repeat domain which binds to a target antigen exposed on the cell surface,
b) a flexible linker,
c) a designed ankyrin repeat domain which binds to the knob of an adenovirus, and
d) a trimerization domain.
wherein said flexible linker is a (Gly₄Ser)₄-linker.

The recombinant adapter molecules of the present disclosure may also comprise a short linker. The short linker is located between the designed ankyrin repeat domain which binds to the knob of an adenovirus and the trimerization domain.

Therefore, in certain embodiments the present disclosure relates to a recombinant adapter molecule comprising from the N- to the C-terminus
a) a designed ankyrin repeat domain which binds to a target antigen exposed on the cell surface,
b) a flexible linker,
c) a short linker, and
d) a trimerization domain.

The short linker does not necessarily be present. Possible short linkers of the present disclosure are linkers which are no longer than four, no longer than three, no longer than two or only one amino acid long. The short linker may also be absent. A preferred short linker is glycine.

### Nucleic Acids, Vectors and Host Cells

The recombinant adapter molecules and the bispecific T cell engagers of the present disclosure are encoded by nucleic acids. Vectors comprising these nucleic acids can be used to transfect cells which express the recombinant adapter molecules and/or the bispecific single chain antibodies. Vectors comprising these nucleic acids can also be used to transfect cells which express the bispecific single chain antibodies, while the recombinant adapter molecules are added as proteins.

Therefore, in certain embodiments, the present disclosure relates to a nucleic acid encoding a recombinant adapter molecule or a bispecific T cell engager of the present disclosure. The present disclosure also relates to a nucleic acid encoding a recombinant adapter molecule comprising
a) a designed ankyrin repeat domain which binds to a target antigen exposed on the cell surface,
b) a designed ankyrin repeat domain which binds to the knob of the adenovirus, and
c) a trimerization domain.

The present disclosure also relates to a nucleic acid encoding bispecific T cell engagers comprising
a) a first binding domain comprising a VH domain and a VL domain that bind to a T cell surface antigen, and
b) a second binding domain comprising a designed ankyrin repeat domain which binds to an epitope of a target antigen exposed on the cell surface. In certain embodiments, said T cell surface antigen is CD3.

In other embodiments, the present disclosure relates to a vector comprising a nucleic acid encoding a recombinant adapter molecule of the present disclosure. The present disclosure also relates to a vector comprising a nucleic acid encoding a recombinant adapter molecule comprising
a) a designed ankyrin repeat domain which binds to a target antigen exposed on the cell surface,
b) a designed ankyrin repeat domain which binds to the knob of the adenovirus, and
c) a trimerization domain.

In other embodiments, the present disclosure relates to a vector comprising a nucleic acid encoding a bispecific T cell engagers of the present disclosure. The present disclosure also relates to a vector comprising a nucleic acid encoding a bispecific T cell engagers comprising
a) a first binding domain comprising a VH domain and a VL domain that bind to a T cell surface antigen, and
b) a second binding domain comprising a designed ankyrin repeat domain which binds to an epitope of a target antigen exposed on the cell surface. In certain embodiments, said T cell surface antigen is CD3.

In another embodiments, the present disclosure relates to a non-oncolytic virus comprising a nucleic acid encoding a recombinant adapter molecule or a bispecific T cell engager of the present disclosure. In another embodiments, the present disclosure relates to an adenovirus comprising a nucleic acid encoding a recombinant adapter molecule or a bispecific T cell engager of the present disclosure. In yet another embodiment, the present disclosure relates to an adenovirus comprising a vector comprising a nucleic acid encoding a recombinant adapter molecule or a bispecific T cell engager of the present disclosure. In certain embodiments said adenovirus carries a TAYT mutation. In certain embodiments said adenovirus carries a HVR7 mutation.

In certain embodiments, the present disclosure also relates to an adenoviral vector comprising a nucleic acid encoding a recombinant adapter molecule comprising
a) a designed ankyrin repeat domain which binds to a target antigen exposed on the cell surface,
b) a designed ankyrin repeat domain which binds to the knob of the adenovirus, and
c) a trimerization domain.

In certain embodiments, the present disclosure also relates to an adenoviral vector comprising a nucleic acid encoding a bispecific T cell engager comprising
a) a first binding domain comprising a VH domain and a VL domain that bind to a T cell surface antigen, and
b) a second binding domain comprising a designed ankyrin repeat domain which binds to an epitope of a target antigen exposed on the cell surface. In certain embodiments, said T cell surface antigen is CD3.

The recombinant adapter molecules and bispecific T cell engagers of the present disclosure can be expressed in prokaryotic cells, such as Escherichia coli, and in eukaryotic cells. Preferred eukaryotic cells are CHO cells. Other preferred eukaryotic cells are HEK293 cells, HEK293-T cells, HEK293-F cells, CHO-S cells and Sf9 cells. Therefore, in certain embodiments the present disclosure provides a eukaryotic cell expressing the recombinant adapter molecules and bispecific T cell engagers of the present disclosure. In certain other the present disclosure provides a CHO cell expressing the recombinant adapter molecules and bispecific T cell engagers of the present disclosure. In certain embodiments, the present disclosure relates to a eukaryotic cell expressing a recombinant adapter molecule comprising
a) a designed ankyrin repeat domain which binds to a target antigen exposed on the cell surface,
b) a designed ankyrin repeat domain which binds to the knob of the adenovirus, and
c) a trimerization domain.

In certain embodiments, the present disclosure relates to a CHO cell expressing a recombinant adapter molecule comprising
a) a designed ankyrin repeat domain which binds to a target antigen exposed on the cell surface,
b) a designed ankyrin repeat domain which binds to the knob of the adenovirus, and
c) a trimerization domain.

### Uses

The non-oncolytic viruses, the recombinant adapter molecules, the trimeric proteins, the bispecific T cell engagers, the nucleic acids, the vectors and the host cells of the present disclosure have numerous uses, such as the use in medicine. Therefore, in certain embodiments the present disclosure provides the recombinant adapter molecules of the present disclosure for use in medicine. In other embodiments the present disclosure provides the bispecific T cell engagers of the present disclosure for use in medicine. In other embodiments the present disclosure provides the nucleic acids encoding the recombinant adapter molecules or the bispecific T cell engagers of the present disclosure for use in medicine. In other embodiments the present disclosure provides the vectors containing the nucleic acids of the present disclosure for use in medicine. In other embodiments the present disclosure provides the adenoviruses containing the recombinant adapter molecules and the bispecific T cell engagers, the nucleic acids or the vectors of the present disclosure for use in medicine.

In certain preferred embodiments, said use in medicine is the use in the treatment of cancer. Therefore, in certain embodiments the present disclosure relates to the recombinant adapter molecules, the trimeric proteins, the bispecific T cell engagers, the nucleic acids, the vectors and the host cells of the present disclosure for use in the treatment of cancer.

In certain embodiments the present disclosure provides a method to treat a patient, said method comprising administering to a patient a non-oncolytic virus of the present disclosure. In certain embodiments the present disclosure provides a method to treat a patient, said method comprising administering to a patient a nucleic acid encoding a recombinant adapter molecule or a bispecific T cell engager of the present disclosure. In certain embodiments, the present disclosure provides a method to treat a patient, said method comprising administering to a patient a vector containing a nucleic acid of the present disclosure. In certain embodiments the present disclosure provides a method to treat a patient, said method comprising administering to a patient a recombinant non-oncolytic virus expressing a recombinant adapter molecule, a bispecific T cell engager, a nucleic acid or a vector of the present disclosure. In certain embodiments the present disclosure provides a method to treat a patient, said method comprising administering to a patient in need thereof a recombinant adenovirus expressing a recombinant adapter molecule, a bispecific T cell engager, a nucleic acid or a vector of the present disclosure.

Principally, the recombinant adapter molecules of the present disclosure, the T cell engagers of the present disclosure, the nucleic acids of the present disclosure, the vectors of the present disclosure, the recombinant non-oncolytic viruses of the present disclosure, and the eukaryotic cells of the present disclosure can be used in the treatment or prevention of any disease or disorder. Preferably, said non-oncolytic virus is an adenovirus.

### CERTAIN EMBODIMENTS OF THE PRESENT DISCLOSURE

1. A recombinant non-oncolytic virus comprising a bispecific T cell engager and a recombinant adapter molecule.

2. The non-oncolytic virus according to claim 1, wherein said bispecific T cell engager comprises
a) a first binding domain comprising a VH domain and a VL domain that bind to a T cell surface antigen, and
b) a second binding domain comprising a designed ankyrin repeat domain which binds to a target antigen exposed on the cell surface.

3. The non-oncolytic virus of claim 2, wherein said T cell surface antigen is CD3.

4. The non-oncolytic virus according to claim 2 or 3, wherein said VH domain of said first binding domain is covalently linked to said VL domain of said first binding domain by a first linker of sufficient length such that said VH domain and said VL domain fold to form a first binding domain that binds to said T cell surface antigen.

5. The non-oncolytic virus according to any one of claims 2-4, wherein said first binding domain and said second binding domain are covalently linked by a second linker of sufficient length such that said first binding domain and said second binding domain fold independently of each other.

6. The non-oncolytic virus according to any one of claims 1-5, wherein said non-oncolytic virus is an adenovirus.

7. The non-oncolytic virus according to claim 6, wherein said adenovirus is of adenovirus serotype 5 or wherein said adenovirus comprises a knob of an adenovirus of serotype 5.

8. The non-oncolytic virus according to claim 7 whereas said adenovirus is a gutless or helper dependent adenovirus.

9. The non-oncolytic virus according to any one of claims 1-8, wherein said bispecific T cell engager is encoded in the genome of the non-oncolytic virus.

10. The non-oncolytic virus according to any one of claims 1-9, wherein said non-oncolytic virus displays said recombinant adapter molecule.

11. The non-oncolytic virus according to any one of claims 1-10, wherein said recombinant adapter molecule comprises
a) a designed ankyrin repeat domain which binds to a target antigen exposed on the cell surface,
b) a designed ankyrin repeat domain which binds to the knob of the adenovirus, and
c) a trimerization domain.

12. The non-oncolytic virus according to claim 11, wherein said trimerization domain is or is derived from the capsid protein SHP of lambdoid phage 21.

13. The non-oncolytic virus according to claim 11 or 12, wherein said trimerization domain comprises the amino acid sequence of SEQ ID No. 1.

14. The non-oncolytic virus according to any one of claims 11-13, wherein said designed ankyrin repeat domain that binds to a knob of an adenovirus comprises the amino acid sequence of SEQ ID No. 2.

15. The non-oncolytic virus according to any one of claims 10-14, wherein said recombinant adapter molecule comprises from the N- to the C-terminus
a) said designed ankyrin repeat domain which binds to a target antigen exposed on the cell surface,
b) said designed ankyrin repeat domain which binds to the knob of the adenovirus, and
c) said trimerization domain.

16. The non-oncolytic virus according to any one of claims 2-15, wherein said first binding domain of said bispecific protein comprises a HCDR1 of SEQ ID No. 3, a HCDR2 of SEQ ID No. 4, a HCDR3 of SEQ ID No. 5, a LCDR1 of SEQ ID No. 6, a LCDR2 of SEQ ID No. 7 and a LCDR3 of SEQ ID No. 8.

17. The non-oncolytic virus according to any one of claims 2-16, wherein said first linker is a glycine-serine linker.

18. The non-oncolytic virus according to any one of claims 2-16, wherein said second linker is a glycine-serine linker.

19. The non-oncolytic virus according to any one of claims 2-18, wherein said target antigen bound by said second binding domain of said bispecific T cell engager and said target antigen exposed on the cell surface and bound by the designed ankyrin repeat domain of said recombinant adapter molecule are the same target antigen.

20. The non-oncolytic virus according to claim 19, wherein said target antigen is HER2 (SEQ ID No. 12).

21. The non-oncolytic virus according to any one of claims 2-18, wherein said target antigen bound by said second binding domain of said bispecific T cell engager and said target antigen exposed on the cell surface and bound by the designed ankyrin repeat domain of said recombinant adapter molecule are different target antigens.

22. The non-oncolytic virus according to any one of claims 2-20, wherein said designed ankyrin repeat domain which binds to a target antigen exposed on the cell surface comprises SEQ ID No. 13.

23. The non-oncolytic virus according to any one of claims 1-22 for use in medicine.

24. The non-oncolytic virus of claim 23, wherein said use in medicine is the use in the treatment of cancer.

25. An eukaryotic cell containing a non-oncolytic virus according to any one of claims 1-22 and/or expressing a bispecific T cell engager encoded on the genome of said non-oncolytic virus.

### EXAMPLES

### Example 1: General Experimental Procedures

### Cell Lines and Blood Samples

BT474 (Cat. No. HTB-20), MCF7 (Cat. No. HTB-22) and SKBR3 (Cat. No. SKBR3) cells were obtained from ATCC and maintained in [R10 Medium (RPMI 1640, 10% FCS, 1% Penicillin-streptomycin) at a density of 0.5 to 2×10⁶ cells/ml].

PBMC's were isolated from healthy adult volunteers. Ethical approval was obtained from the cantonal ethical committee of Zurich, Switzerland (protocol no. KEK-StV-Nr.19/08). Leukocyte concentrate from human donors was acquired from the Blutspende Zurich, Zurich, Switzerland. After Ficoll-Paque (GE Healthcare) gradient separation, donor cells were aliquoted and frozen to be thawed before each assay.

### Viral Vector Generation

The replication-deficient HadV-C5 contains an E1/E3 deletion and 4 mutations in the HVR7 (I421G, T423N, E424S and L426Y) and was generated as previously described (Nat. Commun. 9, 450 (2018)) or ordered from Vector Biolabs (Malvern, PA/USA). The helper-dependent adenovirus containing no adenoviral DNA and harboring the identical 4 mutations was produced as described by Brücher et al. (Mol Ther Methods Clin Dev (2021) 20:572-86). In short, the cell line 116 was transfected with the reporter plasmid containing the HadV-C5 packaging signal and co-transduced with a helper HadV-C5 for replication. Purification was performed via two CsCl gradients at 250,000 g.

### Cloning, Expression and Purification of the Recombinant Adapter Molecules

The recombinant adapter molecules were cloned into the mammalian expression plasmid pcDNA3.1 as previously described (Adv. Cancer Res. 115, 39-67 (2012)). The adapter construct contained an N-terminal HSA leader peptide, an 3C-cleavable His6- and Flag-tag. The retargeting domain is flanked by a BamHI and an HindIII site for ready exchange of the domain. Adapters were expressed in CHO-S cells as described (Protein Expr. Purif. 92, 67-76 (2013)). Following seven days expression, supernatants were 1:1000 dialyzed in PBS pH 7.4, using dialysis tubes with a MWCO cutoff of 12-14 kDa at 4° C. During 24 h, the buffer was exchanged four times 1:10. Dialyzed supernatants were subjected 2.5 ml equilibrated nickel-nitrilotriacetic acid (Ni-NTA) resin (Thermo Fisher) in a PD-10 column (Merck Millipore). All columns were washed with 5 column volumes 20 mM imidazole, 10% glycerol, PBS pH 8.0 and then additionally with 5 column volumes of 500 mM NaCl, 50 mM Tris HCl pH 8.0. The samples were then eluted using 0.7 M imidazole in PBS pH 8.0, followed by subsequent 3C cleavage (GenScript) of the tags during dialysis against 20 mM Hepes at pH 7.4. An additional purification step included an anion exchange chromatography using a MonoQ column (GE Healthcare). Purified protein was dialyzed four times 1:100 in 24 h in endotoxin-free PBS (Merck Millipore) and then shock frozen in liquid nitrogen and stored at −80° C. until usage.

### Expression and Purification of E08-G3

CHO-S cells were diluted in fresh CHOgro medium (4 mM L-glutamine, 0.3% poloxamer 188) at a density of 2×10⁶ cells/mL. 16 h later the cells were resuspended in fresh CHOgro medium (4×10⁶ cells/mL, 250 ml, TubeSpin® Bioreactor 600) and 1.25μg/mL of DNA, 3μg/mL of PEI and 72μg/mL valproic acid were added sequentially with intermitted swirling. Cells were incubated for seven days at 120 rpm, 5% CO2, 31° C. Next, the cells were separated from the supernatant by a centrifugation step (3000 g, 20 min, 4° C.) followed by a filtration step (0.22 μm, Stericup Quick ReleaseGP). Expressed protein was purified by NiNTA beads washed with five column volumes of pH 8.0 PBS supplemented with 20 mM imidazole and 10% glycerol, followed by five column volumes of pH 8.0 TBS containing 50 mM Tris-HCl and 500 mM NaCl. Washed protein was eluted using pH 8.0 PBS supplemented with 500 mM imidazole. Eluted protein samples were incubated together with 3C protease (8 μg/mL) and dialyzed in 20 mM HEPES 20 mM NaCl pH 8.0 (1:8×10⁹ dialysis, 4° C.). The dialyzed protein was then applied to a Mono Q 5/50 GL anion exchange column. Concentration of purified protein samples were determined by measuring the absorbance at 280 nm (NanoDrop™ One Microvolume UV/Vis Spectrophotometer).

### Cell Culture

All human cell lines were cultured in Tissue Culture Flasks 75 cm² with complete RPMI 1640 (10% (v/v) FCS, 1% (v/v) PenStrep, RPMI 1640) at 37° C. and 5% CO2 and all cell counts were determined using a CASY® TT cell counter. 50 ml of buffy coat samples from multiple donors were used for PBMC isolation. Each donor was purified separately by Ficoll gradient centrifugation and subsequently frozen at −80° C. For effector mediated killing assays, PBMCs were prepared one day in advance by resuspension in IL-2-lacking complete RPMI at a cell density of 5×10⁵ cells/mL.

### Effector Cell Mediated Killing Assays

4,000 seeded target cells per well were incubated for 24 h at 37° C. before addition of effector cells. If viral vectors were used, target cells were transduced 6 h after seeding. If purified protein was analyzed, fresh culturing medium with the respective sample and PBMC were added and incubated for 72 h. In case of viral delivered E08-G3, PBMCs were added to without exchange of media. After three days, the supernatant was separated from the adherent cells. The adherent cells were used for the cell viability assay and the supernatant was centrifuged to separate the PBMCs from medium, which in turn was used for the cytokine assay.

### Cell Viability Assay

Cell viability was assayed using XTT (Roche) following the manufacturers protocol. XTT reagent was incubated with the cells for 5 h before measuring absorbance at the Infinite® M1000 instrument (473 nm, reference: 670 nm). Dose response curves were fitted to the XTT data by least squares fit.

### Cytokine Assay

Cytokine assays, including detection of INFγ and IL2, were performed using Human ELISA Kits (ThermoFisher) as described by the manufacturer's instructions. However all measurements were performed using 384-well plates and volumes were reduced accordingly. Absorbance measurements were performed at the Infinite® M1000 instrument.

### Flow Cytometry

For in vitro assays cells were centrifuged at 750 g for 5 min and resuspended in PBS containing 1% BSA and 0.05% azide as well as containing all used antibodies. Cells were then kept at 4° C. in the dark for 30 min and washed twice with PBS. Cells were then resuspended in PBS containing 2% PFA and fixed for 15 min at room temperature. Remaining PFA was then quenched by adding PBS containing 1% BSA and 0.05% azide with a volume of 5 times the fixation volume. The following antibodies were used: CD3 (PerCP-Cy5.5/Biolegend/UCHT1/300429), HER2 (FITC/Thermo Fisher/2G11/BMS120FI).

### Example 2: Constructs of the Present Invention

The present invention is exemplified by making use of recombinant adapter molecules comprising a designed ankyrin repeat domain which binds to a target antigen exposed on the cell surface, a designed ankyrin repeat domain which binds to the knob of the adenovirus, and a trimerization domain.

The present invention is also exemplified by making use of a bispecific T cell engager comprising a scFv that binds to CD3 and a designed ankyrin repeat domain which binds to HER2. This bispecific T cell engager is referred to herein as “E08-G3”.

As designed ankyrin repeat domain which binds HER2 we used the G3 DARPin (Cancer Res (2010) 70, 1595). The amino acid sequence of the G3 DARPin is shown in SEQ ID No. 13. Said binding domain comprising a designed ankyrin repeat domain which binds to an epitope of a target antigen exposed on the cell surface can be present in both, the recombinant adapter molecule and the bispecific T cell engager.

As an exemplary second binding domain comprising a VH domain and a VL domain that bind to CD3, we used a previously described anti-CD3 binding moiety (see U.S. Pat. No. 9,587,021).

This scFv has a VH domain of SEQ ID No. 9 and a VL domain of SEQ ID No. 10. CDRs, according to Kabat, are as follows: HCDR1 (SEQ ID No. 3), HCDR2 (SEQ ID No. 4), HCDR3 (SEQ ID No. 5), LCDR1 (SEQ ID No. 6), LCDR2 (SEQ ID No. 7) and LCDR3 (SEQ ID No. 8). The linker connecting the VH domain and the VL domain has the amino acid sequence of SEQ ID No. 14.

### Example 3: The Bispecific T Cell Engagers are Functionally Potent

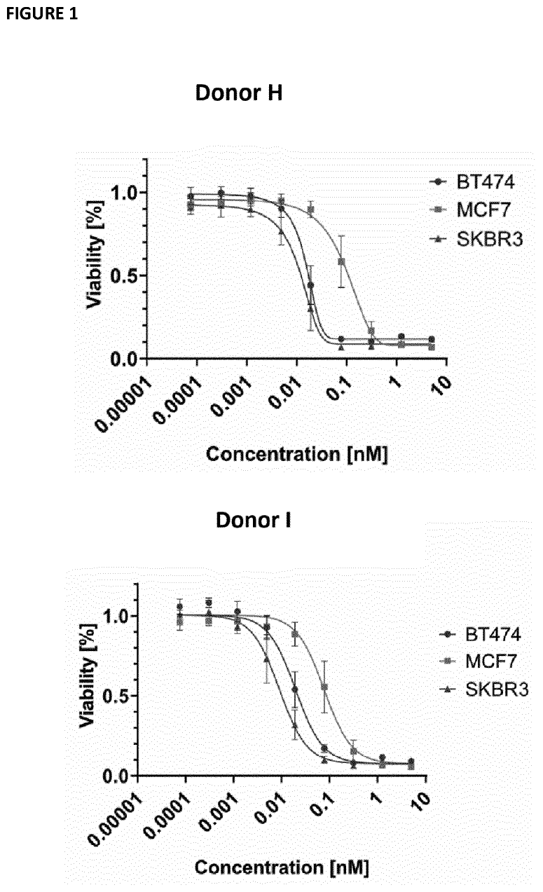

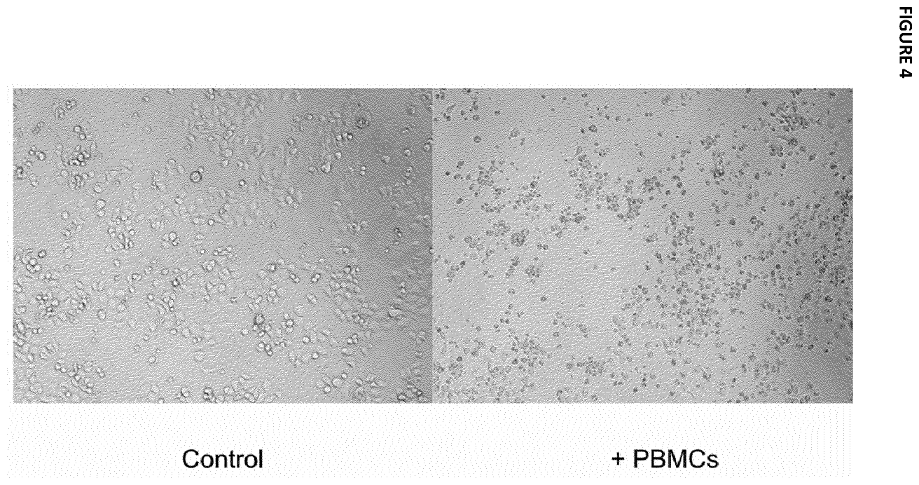

First, the functional activity of the bispecific T cell engagers was tested directly (i.e. without adenoviral delivery) in various cell lines and with PBMC's isolated from healthy donors. Tested was the metabolic activity of the target cells. Results are shown in FIG. 1. The bispecific T cell engager led to a dose-dependent tumor killing on multiple HER2-positive cancer cell lines with multiple donors. The effect is also visible in microscopy, where 200 nM E08-G3 was incubated with SKBR3 cells in the presence of PBMCs or in their absence. Strong killing was only observed if human PBMCs were also present, indicating no toxic effect of E08-G3 alone (FIG. 4).

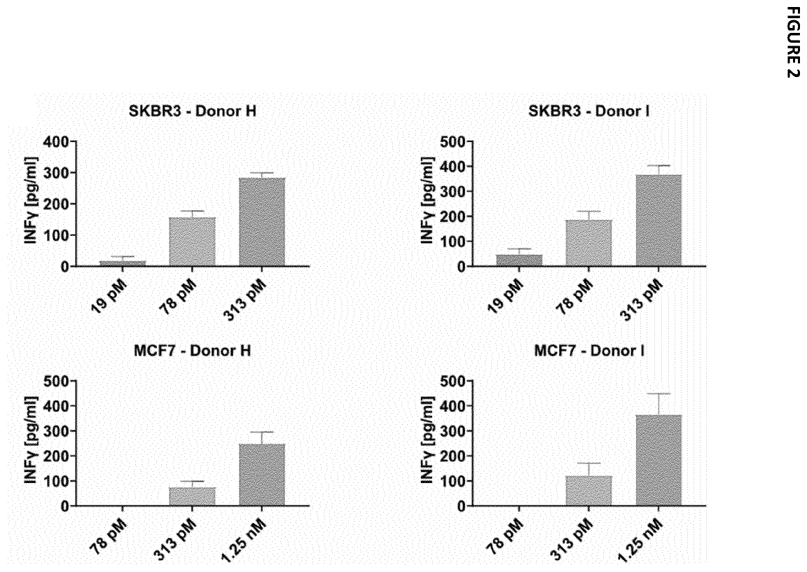

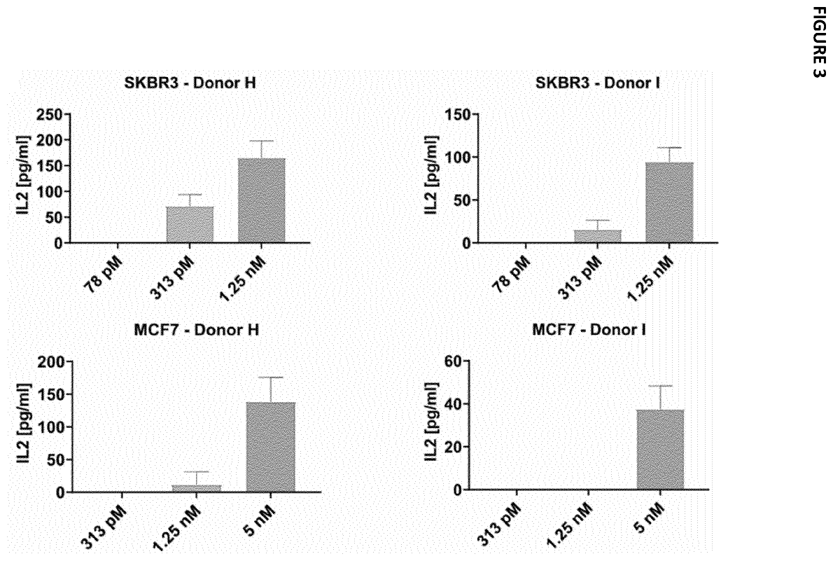

Also, IFNγ and IL2 secretion was measured as a dose dependent response in the presence of PBMCs, a cancer cell line and E08-G3 (FIGS. 2 and 3). It can be clearly seen that the bispecific T cell engagers lead to a dose dependent induction of IFNγ release. IFNγ release is already increasing at single digit picomolar concentrations of the bispecific single-chain antibodies in the presence of target cells. The same effect was also observed for TNFα and perforin secretion (data not shown), and also for additional cell lines (SKOV3 and MCF7).

These results demonstrate that the bispecific T cell engagers are functionally potent. Cytokine release, and the necessity of both, T cells and protein, confirm the effective engagement of T cells.

### Example 4: Adenovirally-Delivered Bispecific T Cell Engagers are Expressed in Target Cells

The high potency and inherent different design of the tumor binding and T cell binding molecule renders E08-G3 as a promising candidate for in situ expression in the tumor microenvironment. We generated high-capacity adenoviral vectors (HC-HAdV-C5) encoding E08-G3 and coated them with our previously validated HER2 targeted adapter molecules (Dreier et al. 2013, Schmid et al 2018). These adapters have been successfully shown to redirect adenoviral vectors to HER2 expressing tumors in vitro and in vivo.

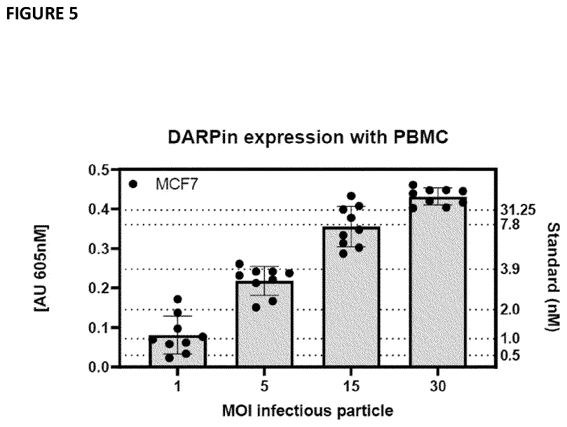

First, we measured successful expression E08-G3 expression in cancer cells upon infection with the retargeted HC-HAdV-C5. Results are shown in FIG. 5. Already at a MOI (multiplicity of infection) of 1 (virus/target cell), the expression of the bispecific T cell engager was clearly detectable. Stronger expression was observed at higher MOI's.

### Example 5: Adenovirally-Delivered Bispecific T Cell Engagers are Functionally Potent

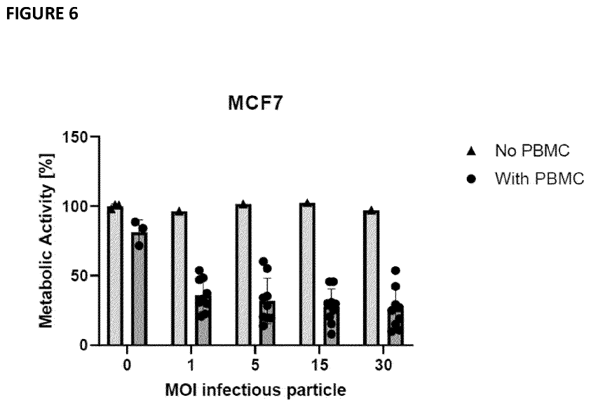

Next, the functional activity of adenoviral delivery bispecific T cell engagers was tested in the MCF7 cell line with and without the addition of PBMCs at various MOI's. Results are shown in FIG. 6. As can be seen, the metabolic activity of the target cells strongly decreased at all MOI's tested. No reduction of metabolic activity was observed in the absence of PBMC's.

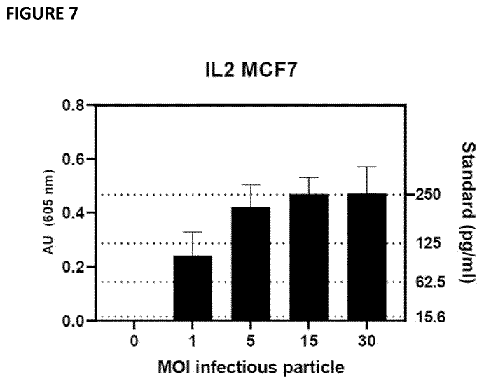

Also the production of IL2 was measured. Results are shown in FIG. 7. Again, IL2 production significantly increased at all MOI's tested, confirming the functional potency of the bispecific T cell engager. The effect observed with the isolated bispecific T cell engager could thus be confirmed in a system of adenoviral delivery.

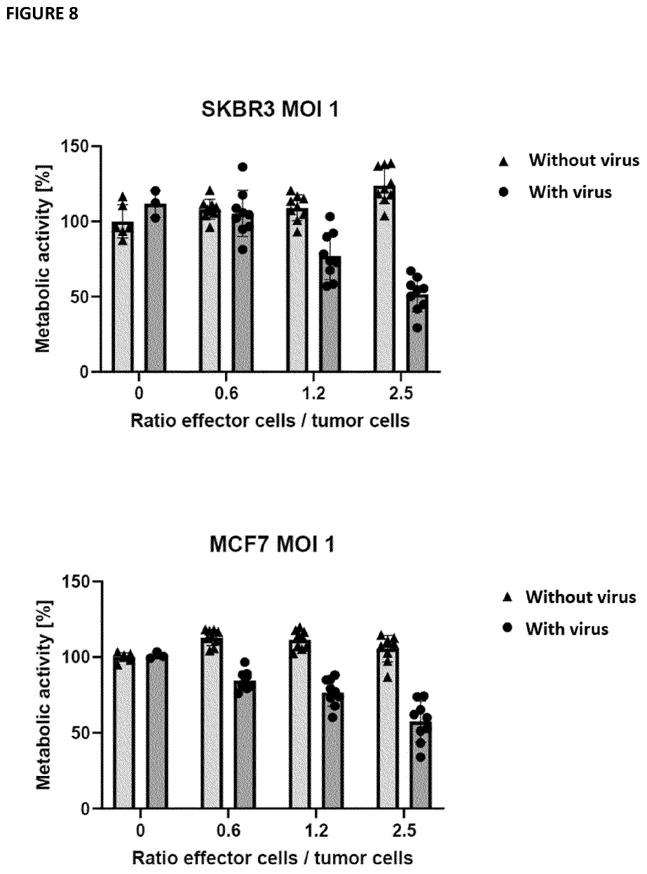

Next, the metabolic activity was measured at a varying ratio of effector cells to tumor cells (i.e. cancer cell line to PBMC'S). Results are shown in FIG. 8 for the cell line SKBR3 (top) and MCF7 (bottom) at an MOI of 1. The bispecific T cell engagers led to a reduction of the metabolic activity of the target cells at a ratio of 1.2 and above of effector cells per tumor cell for the cell line SKBR3, and at a ratio of 0.6 and above effector cells per tumor cell for the cell line MCF7.

These results confirm the activity of adenoviral delivered bispecific T cell engagers of the present disclosure. The bispecific T cell engagers trigger cytokine release and a PBMC-dependent killing of target cells.

### Example 6: Reduction of the Metabolic Rate Correlates with the Killing of HER2-Positive Target Cells

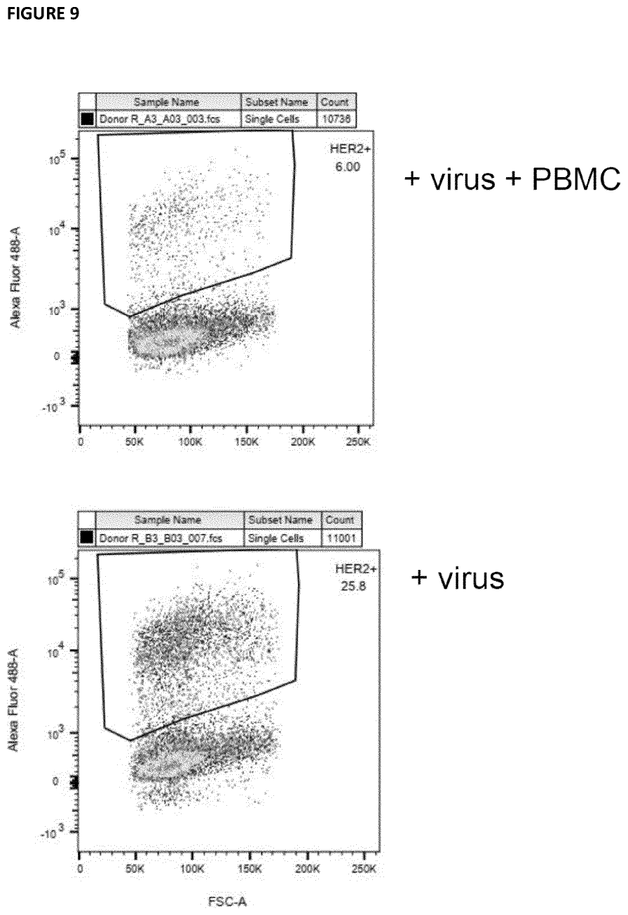

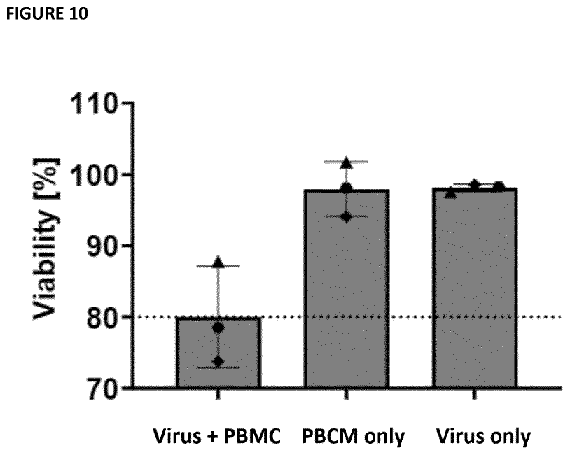

To confirm strict HER2 expression dependence of effector cell induced toxicity, we generated a mixed system of Flp-In-CHO cells expressing human HER2, coculturing them with their parental strain. We could observe a strong reduction of HER2 expression CHO cells by 26-6% if we added retargeted HC-HAdV-C5 encoding E08-G3 (G66) correlating well to a reduction in 20% of their metabolic activity (FIGS. 9 and 10). In contrast, no change in viability was measured using either PBMCs or G66 alone. These results indicate that there is no bystander effect by HER2 retargeted adenoviral vectors delivering E08-G3 in the absence of T cells, further validating the potency of application of the bispecific T cell engagers in HER2 expressing tumor cells. It could also be confirmed that the number of dying cells correlates with the number of HER2-positive cells.

The PBMC-dependent killing induced by the adenoviral-delivered bispecific T cell engager of the present disclosure therefore is directly reflected in the reduced metabolic activity of the target cells.

### Example 7: T Cell Engagers Reduce Tumor Growth and Prolong Survival In Vivo

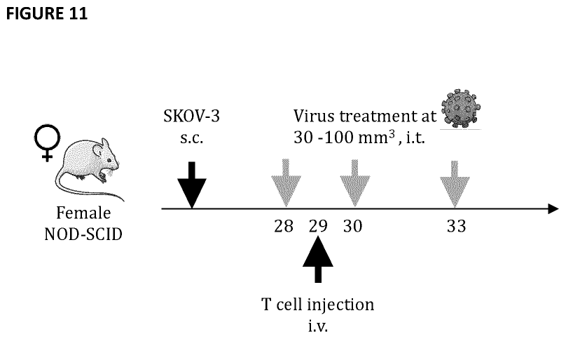

To validate the anti-cancer efficacy of adenovirally-delivered bispecific T cell engagers in vivo, we established a xenograft mice model using female NOD/SCID mice that were injected subcutaneously with 3×10⁶ human ovarian HER-2-expressing SKOV-3 cancer cells. The virus was administered intratumorally once the tumors reached a tumor volume of 30-100 mm³. Each mouse received 3 doses of virus with 1.7×10⁸ transducing units every 2-3 days. Human T cells (7×10⁶) isolated from healthy donors were injected intravenously one day after the first virus administration and 50 μL human IL-2 was given every week intraperitoneally at a dose of 2.75 mg/ml. Tumor-bearing mice injected with T cells only were used as a control. See FIG. 11.

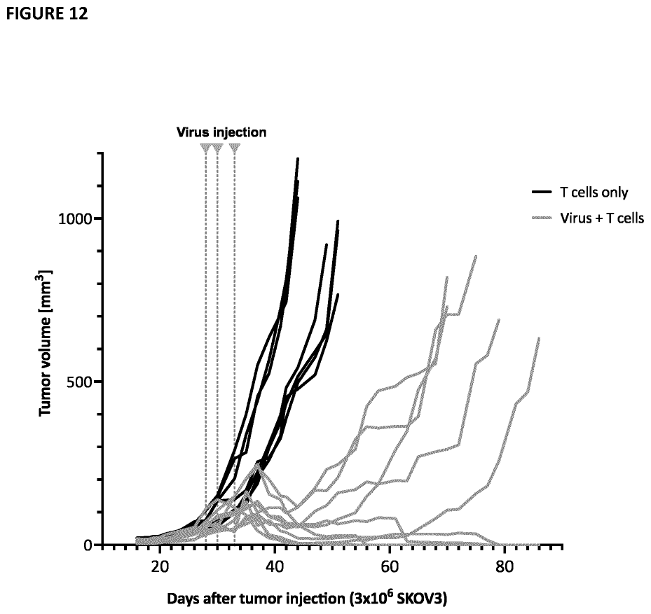

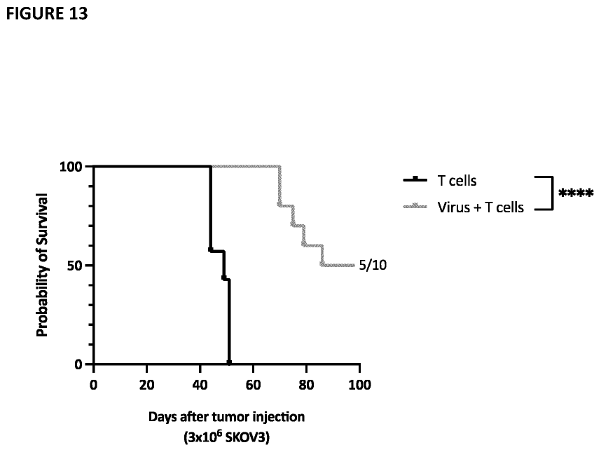

Administration of virus resulted in reduction of tumor growth while control mice showed fast tumor progression. Half of the virus-treated mice achieved complete tumor clearance with no signs of relapse over 80 days after tumor inoculation (FIG. 12). Concomitant with reduced tumor growth, mice treated with virus showed significantly longer survival compared to mice treated with T cells only (FIG. 13). Statistical analysis was done with a Mantel-Cox test (****: p≤0.0001).

These results indicate the anti-cancer efficacy of bispecific T cell engagers in vivo against HER-2 expressing human ovarian cancer cells.

### Example 8: Adenovirally-Delivered T Cell Engagers Leads to Relapse Free Survival in Tumor Bearing Mice

To further validate the anti-cancer efficacy of adenovirally-delivered bispecific T cell engagers in tumor cells, three doses of 1.7×10⁸ transducing units of HER2-retargeted adenoviruses encoding either E08-G3 or GFP, were administered (i.t., 1 mg) into NSG mice bearing subcutaneous tumors of the ovarian cancer cell line SKOV3-HER2. According to prior work this dosage should be sufficient to cover all available HER2.

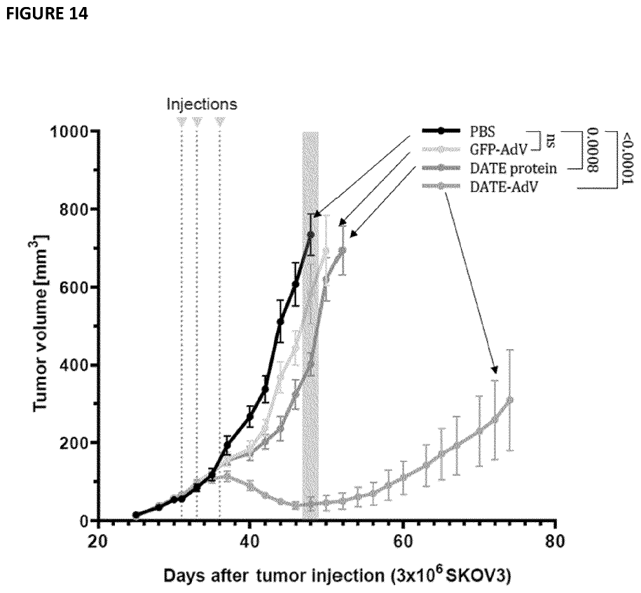

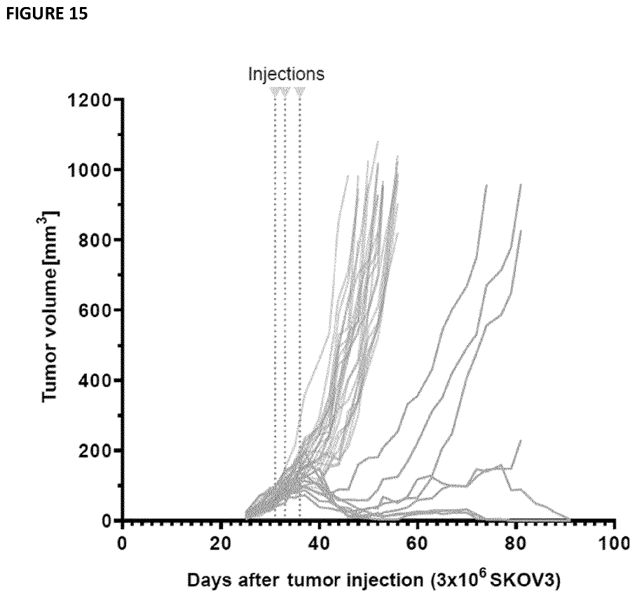

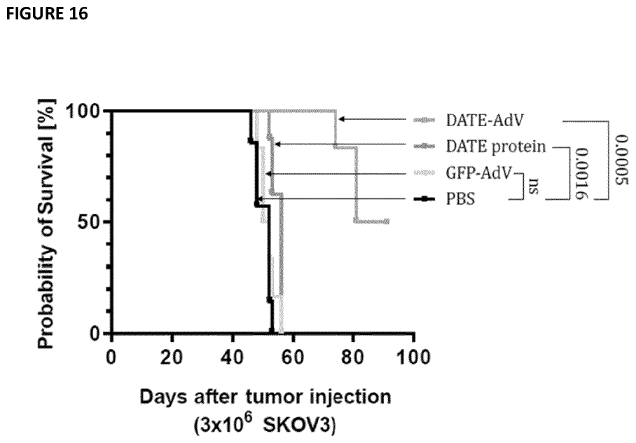

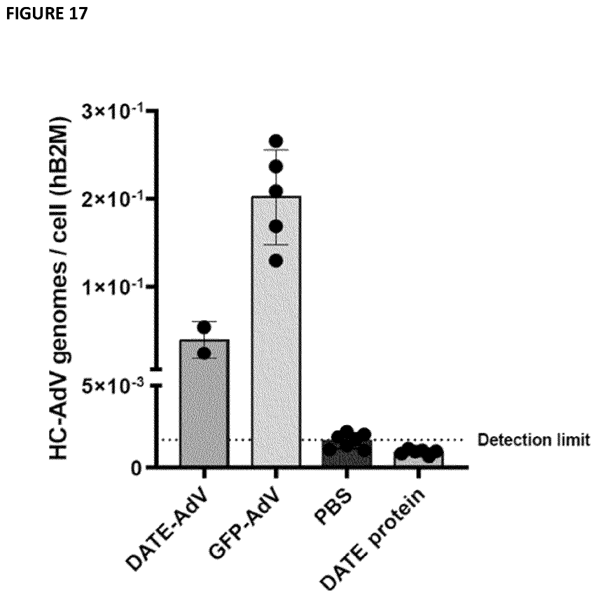

All mice were intravenously (i.v.) reconstituted with 7×10⁶ human T cells isolated from two independent healthy donors. The groups were observed for 91 days post tumor injection for tumor growth. No significant reduction in tumor growth was measured for GFP-AdV-treated mice (FIG. 14). Although injections of purified DARPin-fused T cell engagers (DATEs) were able to delay tumor growth by a minor extent, drastic improvements in outcome were observed upon delivery by adenoviruses encoding DATEs. Furthermore, 50% of mice treated with adenovirally-delivered DATEs went into complete remission and remained tumor-free for 91 days (FIG. 15). This high rate of tumor-free mice was also confirmed with a third human T cell donor (data not shown). Treatment with adenovirally-delivered DATEs furthermore resulted in extended survival indicating prolonged expression of adenovirally-delivered DATEs and improved efficacy by continuous expression (FIG. 16). Both treatments, i.t. injection of recombinant DATEs and adenovirally-delivered DATEs, resulted in detectable accumulation of the T cell engager in the tumor upon sacrifice of the mice, as shown by immunohistochemical analysis (data not shown). Treatment with adenovirally-delivered DATEs increased the presence of T cells, even though the tumor samples were taken around 20 days later. Furthermore, no T cell infiltration was visible by injection of GFP-AdVs although qPCR analysis confirmed successful transduction of cells at the tumor site (FIG. 17). Furthermore, co-localization of T cells and DATEs suggest the induced expansion and infiltration of effector cells.

Altogether, these data show that DARPin-fused T cell engagers are a suitable protein architecture for targeted vector therapy, local secretion, and T cell engagement, with a potent and sustained therapeutic effect in solid tumors.

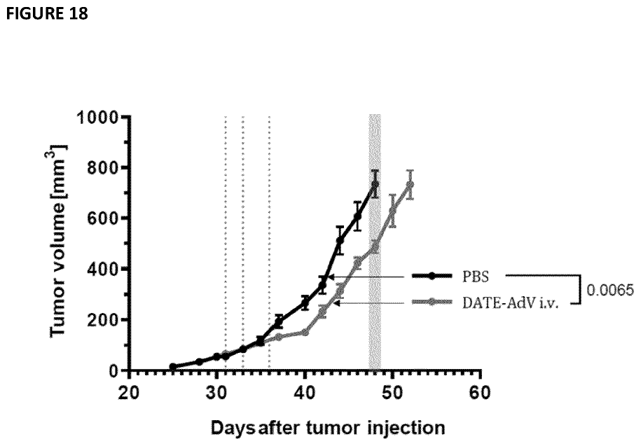

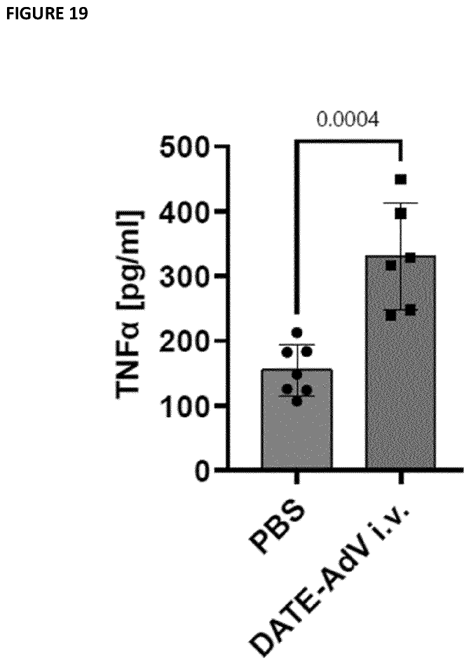

Significant delay in tumor growth was also observed upon i.v. injection of adenovirally-delivered DATEs (FIG. 18). Furthermore, increased proinflammatory TNFα concentrations, secretion and localization of T cell engagers and infiltration by T cells were confirmed by tumor tissue analysis via ELISA (FIG. 19) or immunohistochemistry (data not shown). Neither of the treatments led to significantly elevated alanine aminotransferase levels in the serum compared to the control group, suggesting that no hepatoxicity was induced in this mouse model.

1: A recombinant non-oncolytic virus comprising a bispecific T cell engager and a recombinant adapter molecule, wherein said recombinant adapter molecule comprises
a) a designed ankyrin repeat domain which binds to a target antigen exposed on the cell surface,
b) a designed ankyrin repeat domain which binds to the knob of the adenovirus, and
c) a trimerization domain.

2: The non-oncolytic virus according to claim 1, wherein said bispecific T cell engager comprises
a) a first binding domain comprising a VH domain and a VL domain that bind to a T cell surface antigen, and
b) a second binding domain comprising a designed ankyrin repeat domain which binds to a target antigen exposed on the cell surface.

3: The non-oncolytic virus according to claim 1, wherein said non-oncolytic virus is an adenovirus.

4: The non-oncolytic virus according to claim 1, wherein said bispecific T cell engager is encoded in the genome of the non-oncolytic virus.

5: The non-oncolytic virus according to claim 1, wherein said trimerization domain is or is derived from the capsid protein SHP of lambdoid phage 21.

6: The non-oncolytic virus according to claim 1, wherein said designed ankyrin repeat domain that binds to a knob of an adenovirus comprises the amino acid sequence of SEQ ID No. 2.

7: The non-oncolytic virus according to claim 1, wherein said recombinant adapter molecule comprises from the N- to the C-terminus
a) said designed ankyrin repeat domain which binds to a target antigen exposed on the cell surface,
b) said designed ankyrin repeat domain which binds to the knob of the adenovirus, and
c) said trimerization domain.

8: The non-oncolytic virus according to claim 2, wherein said first binding domain of said bispecific protein comprises a HCDR1 of SEQ ID No. 3, a HCDR2 of SEQ ID No. 4, a HCDR3 of SEQ ID No. 5, a LCDR1 of SEQ ID No. 6, a LCDR2 of SEQ ID No. 7 and a LCDR3 of SEQ ID No. 8.

9: The non-oncolytic virus according to claim 2, wherein said target antigen bound by said second binding domain of said bispecific T cell engager and said target antigen exposed on the cell surface and bound by the designed ankyrin repeat domain of said recombinant adapter molecule are the same target antigen.

10: The non-oncolytic virus according to claim 9, wherein said target antigen is HER2 (SEQ ID No. 12).

11: The non-oncolytic virus according to claim 2, wherein said target antigen bound by said second binding domain of said bispecific T cell engager and said target antigen exposed on the cell surface and bound by the designed ankyrin repeat domain of said recombinant adapter molecule are different target antigens.

12: The non-oncolytic virus according to claim 2, wherein said designed ankyrin repeat domain which binds to a target antigen exposed on the cell surface comprises SEQ ID No. 13.

13: A method for treatment of cancer in a patient, said method comprising administering to the patient the non-oncolytic virus of claim 1.

14: An eukaryotic cell containing a non-oncolytic virus according to claim 1 and/or expressing a bispecific T cell engager encoded on the genome of said non-oncolytic virus.

15: The non-oncolytic virus according to claim 2 wherein said T cell surface antigen is CD3.

16: The non-oncolytic virus of claim 3 wherein the adenovirus is of serotype 5 and/or is a gutless or helper-dependent adenovirus.

17: The non-oncolytic virus of claim 5 wherein said trimerization domain comprises the amino acid sequence of SEQ ID No. 1.


SEQUENCE LISTING (16 sequences)

SEQ ID NO: 1 - protein, 103 aa, Lambdoid phage 21
VRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 2 - protein, 157 aa, synthetic construct
RSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQG

SEQ ID NO: 3 - protein, 5 aa, synthetic construct
TYAMN

SEQ ID NO: 4 - protein, 19 aa, synthetic construct
RIRSKYNNYATYYADSVKD

SEQ ID NO: 5 - protein, 14 aa, synthetic construct
HGNFGNSYVSWFAY

SEQ ID NO: 6 - protein, 14 aa, synthetic construct
RSSTGAVTTSNYAN

SEQ ID NO: 7 - protein, 7 aa, synthetic construct
GTNKRAP

SEQ ID NO: 8 - protein, 9 aa, synthetic construct
ALWYSNLWV

SEQ ID NO: 9 - protein, 127 aa, synthetic construct
GSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 10 - protein, 112 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGKL

SEQ ID NO: 11 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 12 - protein, 1255 aa, Homo sapiens [sequence omitted]

SEQ ID NO: 13 - protein, 121 aa, synthetic construct
DLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQ

SEQ ID NO: 14 - protein, 20 aa, synthetic construct
GGGGSGGGGSGGGGSGGGGS

SEQ ID NO: 15 - protein, 259 aa, synthetic construct
GSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGKL

SEQ ID NO: 16 - protein, 402 aa, synthetic construct
GSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGKLGGGGSGGGGSGGGGSGGGGSGRDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQ